(19) United States
(12) Patent Application Publication
(10) Pub. No.: US 2005/0169941 A1
(43) Pub. Date: Aug. 4, 2005

(54) Use of amino-oxy functional groups in the preparation of protein-polysaccharide conjugate vaccines

(76) Inventor: Andrew Lees, Silver Spring, MD (US)

Correspondence Address: Min, Hsieh & Hack LLP, c/o PortfoliolP, P.O. Box 52050, Minneapolis, MN, 55402, US

(21) Appl. No.: 11/044,866

(22) Filed: Jan. 27, 2005

Related U.S. Application Data
(60) Provisional application No. 60/539,573, filed on Jan. 29, 2004.
(60) Provisional application No. 60/589,019, filed on Jul. 20, 2004.

(51) Int. Cl. C07K 14/195; A61K 39/385; A61K 39/02

(52) U.S. Cl. 424/234.1; 530/395

(57) ABSTRACT

The invention relates to a process for preparing a conjugate comprising combining an amino-oxy homofunctional or heterofunctional reagent with an entity chosen from polysaccharides, oligosaccharides, carbohydrates, and carbohydrate-containing molecules containing at least one carbonyl group, to form a polysaccharide, oligosaccharide, carbohydrate, or carbohydrate-containing molecule functionalized via at least one oxime linkage. The functionalized compound is then reacted either directly or indirectly with a protein moiety to form a protein-carbohydrate conjugate that may be used as a vaccine.

This application claims benefit of priority of U.S. Provisional Application Nos. 60/539,573 filed Jan. 29, 2004, and 60/589,019, filed Jul. 20, 2004.

### BACKGROUND OF THE INVENTION

The present invention relates to a process of covalently linking proteins and polysaccharides to form conjugate vaccines comprising a reaction between carbonyl-containing groups and amino-oxy functional groups.

In the process of vaccination, medical science uses the body's innate ability to protect itself against invading agents by immunizing the body with antigens that will not cause the disease but will stimulate the formation of antibodies that will protect against the disease. For example, dead organisms are injected to protect against bacterial diseases such as typhoid fever and whooping cough, toxoids are injected to protect against tetanus and diptheria, and attenuated organisms are injected to protect against viral diseases such as poliomyelitis and measles.

It is not always possible, however, to stimulate antibody formation merely by injecting the foreign agent. The vaccine preparation must be immunogenic, that is, it must be able to induce an immune response. Certain agents such as tetanus toxoid can innately trigger the immune response, and may be administered in vaccines without modification. Other important agents are not immunogenic, however, and must be converted into immunogenic molecules or constructs before they can induce the immune response.

The immune response is a complex series of reactions that can generally be described as follows: (1) the antigen enters the body and encounters antigen-presenting cells that process the antigen and retain fragments of the antigen on their surfaces; (2) the antigen fragments retained on the antigen-presenting cells are recognized by T cells that provide help to B cells; and (3) the B cells are stimulated to proliferate and divide into antibody forming cells that secrete antibody against the antigen.

Most antigens only elicit antibodies with assistance from the T cells and, hence, are known as T-dependent (TD). Examples of such T-dependent antigens are tetanus and diphtheria toxoids.

Some antigens, such as polysaccharides, cannot be properly processed by antigen presenting cells and are not recognized by T cells. These antigens do not require T cell assistance to elicit antibody formation but can activate B cells directly and, hence, are known as T-independent antigens (TI). Such T-independent antigens include H. influenzae type b polyribosyl-ribitol-phosphate (PRP) and pneumococcal capsular polysaccharides.

There are other differences between T-independent and T-dependent antigens.
A) T-dependent antigens, but not T-independent antigens, can prime an immune response so that a memory response results on secondary challenge with the same antigen.
B) The affinity of the antibody for antigen increases with time after immunization with T-dependent, but not T-independent antigens.
C) T-dependent antigens stimulate an immature or neonatal immune system more effectively than T-independent antigens.
D) T-dependent antigens usually stimulate IgM, IgG1, IgG2a, and IgE antibodies, while T-independent antigens stimulate IgM, IgG1, IgG2b, and IgG3 antibodies.

T-dependent antigens can stimulate primary and secondary responses, which are long-lived in both adult and in neonatal immune systems, but must frequently be administered with adjuvants (substances that enhance the immune response). Very small proteins, such as peptides, are rarely immunogenic, even when administered with adjuvants.

T-independent antigens, such as polysaccharides, are able to stimulate immune responses in the absence of adjuvants, but cannot stimulate high level or prolonged antibody responses. They are also unable to stimulate an immature or B cell defective immune system (Mond, J. J., Immunological Reviews, 64: 99 (1982); Mosier, D. E. et al., J. Immunol., 119: 1874 (1977)).

For T-independent antigens, it is desirable to provide protective immunity against such antigens to children, especially against capsular polysaccharides found on organisms such as H. influenzae, S. pneumoniae, and Neisseria meningiditis.

One approach to enhance the immune response to T-independent antigens involves conjugating polysaccharides such as H. influenzae PRP (Cruse, J. M., Lewis, R. E. Jr., eds., Conjugate Vaccines in Contributions to Microbiology and Immunology, Vol. 10, (1989)), or oligosaccharide antigens (Anderson, P. W. et al., J. Immunol., 142: 2464, (1989)) to a T-dependent antigen such as tetanus or diphtheria toxoid. Recruitment of T cell help in this way has been shown to provide enhanced immunity to many infants that have been immunized.

Protein-polysaccharide conjugate vaccines stimulate an anti-polysaccharide antibody response in infants who are otherwise unable to respond to the polysaccharide alone.

Conjugation of a protein and a polysaccharide may provide other advantageous results. For example, Applicant has found that a protein/polysaccharide conjugate may enhance the antibody response not only to the polysaccharide component, but also to the protein component. This effect is described, for example, in U.S. Pat. No. 5,955,079. This effect also is described in A. Lees, et al., Vaccine, 12(13): 1160 (1994).

Techniques have been developed to facilitate coupling of proteins and polysaccharides. See, for example, Dick, W. E. et al., “Glyconjugates of Bacterial Carbohydrate Antigens: A Survey and Consideration of Design and Preparation Factors,” Conjugate Vaccines (Eds. Cruse, et al.), p. 48 (1989). Many techniques for activation of carbohydrates, however, are not suitable for use in aqueous media because the activating or functional reagents are not stable in water. For example, N,N′-carbonyldiimidazole, as described in Marburg et al., U.S. Pat. No. 4,695,624, must be used in organic media.

Homofunctional and heterofunctional vinylsulfone reagents have been used to activate polysaccharides. The activated polysaccharides are reacted with a protein, peptide, or hapten, under appropriate reaction conditions, to produce the conjugate. This is described in more detail in U.S. Pat. No. 6,309,646. Another method for producing conjugate vaccines comprises mixing a uronium salt reagent with a soluble first moiety, such as a polysaccharide or carbohydrate, and combining therewith a second moiety, such as a protein, peptide, or carbohydrate, to form the conjugate vaccine. This method is described in U.S. Pat. No. 6,299,881.

Most carbohydrates must be activated before conjugation, and cyanogen bromide (CNBr) is frequently the activating agent of choice. See, e.g., Chu et al., Inf. & Imm., 40: 245 (1983). The first licensed conjugate vaccine was prepared with CNBr to activate HIB PRP, which was then derivatized with adipic dihydrazide and coupled to tetanus toxoid using a water-soluble carbodiimide.

The use of 1-cyano-4-(dimethylamino)-pyridinium tetrafluoroborate, also called “CDAP,” has been described for use in aqueous media to activate polysaccharides. These activated polysaccharides may be directly or indirectly coupled to proteins. The use of CDAP is described in, for example, U.S. Pat. No. 5,849,301 and in Lees, et al., “Activation of Soluble Polysaccharides with 1-Cyano-4-Dimethylamino Pyridinium Tetrafluoroborate For Use in Protein-Polysaccharide Conjugate Vaccines and Immunological Reagents,” Vaccine, 14(3): 190 (1996).

To briefly summarize the CNBr-activation method, CNBr is reacted with the carbohydrate at a high pH, typically a pH of 10 to 12. At this high pH, cyanate esters are formed with the hydroxyl groups of the carbohydrate. These, in turn, are reacted with a bifunctional reagent, commonly a diamine or a dihydrazide. These derivatized carbohydrates may then be conjugated via the bifunctional group. In certain limited cases, the cyanate esters may also be directly reacted to protein.

The high pH is necessary to ionize the hydroxyl group because the reaction requires the nucleophilic attack of the hydroxyl ion on the cyanate ion (CN⁻). As a result, CNBr produces many side reactions, some of which add neo-antigens to the polysaccharides. Wilcheck, M. et al., Affinity Chromatography. Meth. Enzymol., 104: 3-55 (1984). More importantly, many carbohydrates or moieties such as Hib, PRP, and capsular polysaccharides from and pneumococcal type 6 and Neisseria meningitis A can be hydrolyzed or damaged by the high pH necessary to perform the cyanogen bromide activation.

Another problem with the CNBr activation method is that the cyanate ester formed is unstable at high pH and rapidly hydrolyzes, reducing the yield of derivatized carbohydrate and, hence, the overall yield of carbohydrate conjugated to protein. Many other nonproductive side reactions, such as those producing carbamates and linear imidocarbonates, are promoted by the high pH. This effect is described in Kohn et al., Anal. Biochem, 115: 375 (1981). Moreover, CNBr itself is highly unstable and spontaneously hydrolyzes at high pH, further reducing the overall yield.

Protein-polysaccharide conjugate vaccines may also be formed via reductive amination. In this method, aldehydes on the polysaccharide are reacted with amines on the protein to form a reversible Schiff base. The Schiff base is subsequently reduced to form a stable linkage between the amine and the aldehyde. This process is beset by a number of problems. The formation of the Schiff base is slow and inefficient, and the overall reaction is further impeded by the large size of the two components (i.e., the polysaccharide and protein), which need to be in close proximity with each other in order to react. In order to overcome this problem, the polysaccharide is often broken down into oligosaccharides prior to coupling.

The use of dimethylsulfoxide (DMSO) promotes the formation of the Schiff base, but this organic solvent can harm the protein. Sometimes a multistep protocol is used, in which a spacer group (e.g., hexane diamine or adipic dihydrazide) is added to the polysaccharide via reductive amination, and this spacer is subsequently ligated to the protein. Using a high concentration of the spacer helps to force the reaction and increase the yield. Elevated temperatures and prolonged reaction times are also used to promote the reaction. However, these can also be detrimental to the protein and the polysaccharide. Furthermore, as amines must be deprotonated to react with aldehydes, the Schiff base formation usually requires the use of alkaline solutions, i.e., solutions at a pH≧8. Prolonged reactions at elevated temperature and pH can be detrimental to both the protein and the polysaccharide. Furthermore, the reductive step, which usually involves the use of cyanoborohydride or pyridine-boranes, can be inefficient and deleterious to the protein. Also, these reagents can be hazardous to work with in large quantities. A further limitation of the reductive amination method is the highly random nature of the linkage sites between the protein and the polysaccharide.

Accordingly, there remains a need in the art for an efficient and effective process for preparing conjugate vaccines.

### SUMMARY OF THE INVENTION

One embodiment includes a process for preparing a conjugate vaccine, comprising:
(a) reacting a first moiety containing at least one carbonyl-containing group with at least one amino-oxy reagent to form at least one pendent functional group on the first moiety, wherein the first moiety is chosen from polysaccharides, oligosaccharides, carbohydrates, and carbohydrate-containing molecules;
(b) reacting the first moiety containing at least one pendent functional group with a second moiety to form a composition comprising a conjugate, wherein the second moiety is chosen from proteins, peptides, and haptens; and
(c) combining the conjugate with a pharmaceutically acceptable delivery vehicle to form a conjugate vaccine.

Another embodiment includes a process for preparing a conjugate vaccine, comprising:
(a) reacting a first moiety containing at least one pendent amino-oxy group with a second moiety to form a composition comprising a conjugate,
(b) wherein the first moiety is chosen from polysaccharides, oligosaccharides, carbohydrates, and carbohydrate-containing molecules, and the second moiety is chosen from proteins, peptides, and haptens; and
(c) combining the conjugate with a pharmaceutically acceptable delivery vehicle to form a conjugate vaccine.

Another embodiment includes a process for preparing a conjugate vaccine, comprising:
(a) reacting a first moiety chosen from polysaccharides, oligosaccharides, carbohydrates, and carbohydrate-containing molecules, with
(b) a second moiety reacted with at least one amino-oxy reagent, wherein the second moiety is chosen from proteins, peptides, and haptens, to form a composition comprising a conjugate; and
(c) combining the conjugate with a pharmaceutically acceptable delivery vehicle to form a conjugate vaccine.

Yet another embodiment includes a process for preparing a conjugate vaccine, comprising:
(a) reacting a first moiety with a second moiety containing at least one pendent amino-oxy group to form a composition comprising a conjugate,
wherein the first moiety is chosen from polysaccharides, oligosaccharides, carbohydrates, and carbohydrate-containing molecules, and the second moiety is chosen from proteins, peptides, and haptens; and
(b) combining the conjugate with a pharmaceutically acceptable delivery vehicle to form a conjugate vaccine.

A further embodiment includes a process for preparing a conjugate vaccine, comprising:
(a) providing a first moiety chosen from polysaccharides, oligosaccharides, carbohydrates, and carbohydrate-containing molecules;
(b) providing a second moiety chosen from N-terminal 1,2-aminoalcohols which can be oxidized to contain at least one aldehyde group;
(c) functionalizing said second moiety with at least one amino-oxy reagent;
(d) reacting said first moiety with the functionalized second moiety to form a composition comprising a conjugate; and
(e) combining the conjugate with a pharmaceutically acceptable delivery vehicle to form a conjugate vaccine.

A further embodiment includes a process for preparing a conjugate vaccine, comprising:
(a) reacting a first moiety containing at least one pendent amino-oxy group, wherein the first moiety is chosen from polysaccharides, oligosaccharides, carbohydrates, and carbohydrate-containing molecules;
(b) reacting the first moiety with a second moiety to form a composition comprising a conjugate, wherein the second moiety is chosen from glycoproteins containing at least one carbonyl group; and
(c) combining the conjugate with a pharmaceutically acceptable delivery vehicle to form a composition comprising an conjugate vaccine.

Still another embodiment includes a process for preparing a conjugate vaccine, comprising:
(a) reacting a first moiety chosen from polysaccharides, oligosaccharides, carbohydrates, and carbohydrate-containing molecules with a second moiety chosen from proteins, peptides, and haptens to form a composition comprising a conjugate,
(b) wherein the first moiety contains at least one reducing end derivatized with an amino-oxy reagent, and
(c) combining the conjugate with a pharmaceutically acceptable delivery vehicle to form a conjugate vaccine.

### BRIEF DESCRIPTION OF THE DRAWINGS

FIG. 1 is an SDS-page chromatogram showing a high degree of protein-polysaccharide conjugation.

FIG. 2 is an SDS-page chromatogram showing BSA-polysaccharide conjugation.

FIG. 3 shows the results of a resorcinol assay for protein and carbohydrate of fractions eluting from an S-400HR™ (Pharmacia) gel filtration column.

FIG. 4 shows an SDS-PAGE chromatogram indicating the occurrence of protein-polysaccharide conjugation.

FIGS. 5A-5D indicate the presence of higher molecular weight conjugates of fractions eluting from an S-400HR™ (Pharmacia) gel filtration column.

FIG. 6 is an SDS-PAGE chromatogram showing the presence of conjugate fractions.

FIG. 7 is a chromatogram comparing a conjugate with its unconjugated components.

FIG. 8 illustrates the results of an opsonic assay.

### DEFINITIONS

Amino-oxy reagent refers to a reagent with the structure NH₂—O—R. R can be any group capable of bonding to the amino-oxy nitrogen. According to one aspect of the disclosure, R is a functional group, e.g., an amine, thiol, or other chemical group facilitating coupling to, e.g., a protein.

Conjugate means to chemically link or join together.

Functionalize means to add at least one group that facilitates further reaction. Typical functional groups include amino-oxy, thiol, maleimide, halogen, haloacyl, aldehyde, hydrazide, hydrazine, and carboxyl. Other functional groups would be well known to the person of ordinary skill in the art and can be found discussed in Hermanson, Bioconjugation Techniques.

Hapten refers to a small molecule such as a chemical entity that by itself is not able to elict an antibody response, but can elicit an antibody response once it is coupled to a carrier.

Homofunctional, when discussing an amino-oxy reagent, refers to a reagent that has at least two amino-oxy functional groups. The homofunctional agent may be homobifunctional or homomultifunctional, i.e., having two, three, four or more amino-oxy functional groups.

Heterofunctional, when discussing an amino-oxy reagent, refers to a reagent that has at least one amino-oxy functional group and at least one other non-amino-oxy functional group. The heterofunctional agent may be heterobifunctional or heteromultifunctional, i.e., having two, three, four or more amino-oxy functional groups. It may also have more than one other non-amino-oxy functional group, such as two, three, or four or more, of either the same type or different types.

Moiety refers to one of the parts of a conjugate.

Pendent functional group refers to a functional group that is exists on or is exposed on a molecule.

Spacer refers to an additional molecule that is used to indirectly couple the first moiety to the second moiety.

### DETAILED DESCRIPTION OF THE INVENTION

A. Strategy for Conjugation

The present invention provides an alternative to prior art processes for preparing conjugate vaccines. Specifically, the invention provides for new methods of conjugating a first moiety to a second moiety, where the first moiety is chosen from polysaccharides, oligosaccharides, carbohydrates, and carbohydrate-containing molecules and the second moiety is chosen from proteins, peptides, and haptens, and the conjugation proceeds using at least one amino-oxy functional group.

There are a number of ways of reacting the first and second moiety within the scope of the invention and each of these methods rely on using at least one amino-oxy group in the process.
1. At least one amino-oxy reagent with one amino-oxy group may be reacted with the first moiety to form a composition with at least one non-amino-oxy pendent functional group.
2. At least one amino-oxy reagent with more than one amino-oxy group may be reacted with the first moiety to form a composition with at least one amino-oxy pendent functional group. In this embodiment, there may optionally additionally be present at least one non-amino-oxy pendent functional group.
3. At least one amino-oxy reagent with one amino-oxy group may be reacted with the second moiety to form a composition with at least one non-amino-oxy pendent functional group.
4. At least one amino-oxy reagent with more than one amino-oxy group may be reacted with the second moiety to form a composition with at least one amino-oxy pendent functional group. In this embodiment, there may optionally additionally be present at least one non-amino-oxy pendent functional group.

Thus, in this invention, at least one of the first moiety and the second moiety will be reacted with an amino-oxy reagent, and will result in a composition with at least one pendent functional group (at least one of an amino-oxy or non-amino-oxy pendent functional group). It is possible to functionalize both the first moiety and the second moiety according to any combination of strategies 1 or 2 (first moiety) and 3 or 4 (second moiety), as set forth immediately above. In another embodiment, either the first moiety or the second moiety may be functionalized.

The first moiety and the second moiety may then be conjugated together. This conjugation may proceed directly, by linking the pendent functional group on the first moiety directly to the second moiety. Alternatively, this conjugation may proceed indirectly, by linking the pendent functional group on the first moiety to an additional agent called a spacer, which is then linked to the second moiety. Certainly, a similar strategy may be followed with a pendent functional group on the second moiety, simply by reversing the positions of the first and second moiety.

B. The First Moiety: Polysaccharide, Oligosaccharide, Carbohydrate, and Carbohydrate-Containing Molecules

As used herein, “carbohydrate” means any soluble monosaccharide, disaccharide, oligosaccharide, or polysaccharide. Examples of suitable polysaccharides for use in the process of the invention include bacterial, fungal, and viral polysaccharides. Soluble polysaccharides (i.e., polysaccharides present in solution), such as water-soluble polysaccharides, are suitable for use in accordance with the present invention. Specific examples of suitable polysaccharides include Salmonella typhi Vi antigen; Neisseria meningiditis polysaccharide C; and Pneumococcal polysaccharides, such as Pneumococcal polysaccharide type 14

According to certain embodiments of the present invention, the carbohydrate is naturally occurring, a semisynthetic, or a totally synthetic large molecular weight molecule. According to one embodiment, at least one carbohydrate-containing moiety is selected from E. coli polysaccharides, S. aureus polysaccharides, dextran, carboxymethyl cellulose, agarose, Pneumococcal polysaccharides (Pn), Ficoll, Cryptococcus neoformans, Haemophilus influenzae PRP, P. aeroginosa, S. pneumoniae, Group A and B streptococcus, N. meningitidis, and combinations thereof.

According to one embodiment, the carbohydrate-containing moiety is a dextran. As used herein, “dextran” (dex) refers to a polysaccharide composed of a single sugar, which may be obtained from any number of sources (e.g., Pharmacia). Another preferred carbohydrate-containing moiety is Ficoll, which is an inert, semisynthetic, non-ionized, high molecular weight polymer. Additional non-limiting examples of moieties that may be used in accordance with the present invention include lipopolysaccharides (“LPS”), lipooligopolysaccharides (“LOS”), lipotechoic acid (“LTA”), deaceylated LPS, deaceylated LTA, delipidated LPS, delipidated LTA, and related molecules. Generally, a carbohydrate-containing molecule that has been coupled using reductive amination requires the formation of an aldehyde moiety. In those instances, for example, these aldehydes may also be coupled using amino-oxy chemistry described herein.

Reductive amination has been used to couple LPS and LOS, both of which can be coupled using amino-oxy chemistry. Examples of coupling of LPS and LOS using reductive amination chemistry may be found in Mieszala et al., Carbohydrate Research, 338: 167 (2003); Jennings et al., Inf. & Immun., 43: 407 (1984); and U.S. Pat. No. 4,663,160.

C. The Second Moiety: Proteins, Peptides and Haptens

In accordance with the present invention, various different proteins can be coupled to various different polysaccharides. The following list includes examples of suitable proteins that may be used in accordance with the invention: viral proteins, bacterial proteins, fungal proteins, parasitic proteins, animal proteins. Glycoproteins from any of the above sources may also be used to form a conjugate with the first moiety. Lipids, glycolipids, peptides, and haptens are also suitable for use as a second moiety in this invention. Haptenated proteins, i.e., proteins derivatized with haptens, are also suitable for use as a second moiety in this invention.

Specific proteins include tetanus toxoid (TT), pertussis toxoid (PT), bovine serum albumin (BSA), lipoproteins, diptheria toxoid (DT), heat shock protein, T-cell superantigens, protein D, CRM197, and bacterial outer-membrane protein. All of these protein starting materials may be obtained commercially from biochemical or pharmaceutical supply companies (e.g., American Tissue Type Collection in Rockville, Md. or Berna Laboratories of Florida) or may be prepared by standard methodologies, such as those described in J. M. Cruse and R. E. Lewis (Eds.), “Conjugate Vaccines in Contributions to Microbiology and Immunology”, Vol. 10 (1989).

D. Methods for Functionalizing the First or Second Moiety with an Amino-Oxy Group

The amino-oxy (also referred to as oxy-amine, amino-oxy, aminooxy, and amino-oxy) functional group, NH₂—O—R, has a lower pKa than the amines found on proteins, and is nucleophilic at much lower pH. Amino-oxy groups react well with carbonyl-containing groups, e.g., aldehydes and ketones, to form highly stable oximes. The optimum pH for the reaction can range from 4 to 8, for example from 5 to 7. According to one aspect of the invention, the optimum pH is around 5. Since oximes are stable, the reductive step in the reductive amination process, discussed above, is optional. The high efficiency of the reaction may result in shorter reaction times. Furthermore, it is possible to exert some control over the reaction sites between the complementary reagents. By contrast, the reaction of hydrazides and amines with groups such as, for example, ketones, is slower and far less efficient.

The protein and polysaccharide are functionalized with complementary oxime-forming groups, and reacted to form oxime-linked protein-polysaccharide conjugate vaccines. According to one aspect of the invention, the protein is directly linked to the polysaccharide.

According to one embodiment, there is provided a process comprising combining an amino-oxy homofunctional or heterofunctional reagent with an entity chosen from polysaccharides, oligosaccharides, carbohydrates, and carbohydrate-containing molecules containing at least one carbonyl group, to form a polysaccharide, oligosaccharide, carbohydrate, or carbohydrate-containing molecule functionalized via at least one oxime linkage. Functionalized means to add a group which facilitates further reaction, for example, thiol, carboxy, amino-oxy, halogen, aldehydes, and the like. This embodiment may be illustrated by the following non-limiting illustration (“Ps” denotes a polysaccharide): 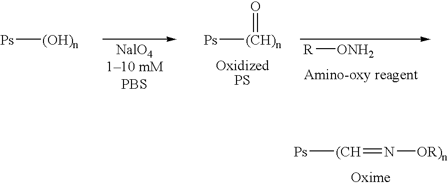
R is a functional group, e.g., an amino-oxy, amine, thiol, or other chemical group, such as those listed below, for facilitating coupling to the protein:

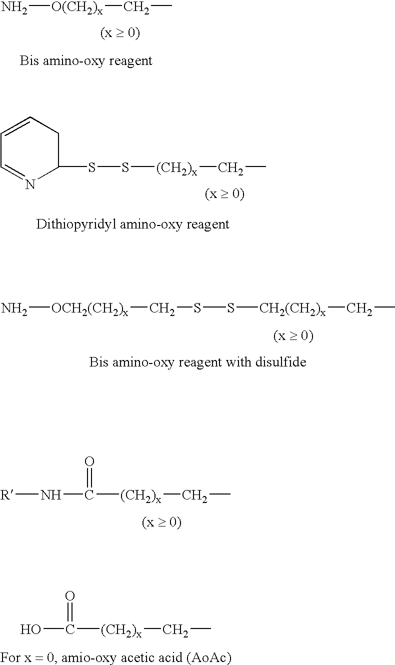

The at least one pendent functional group is then reacted directly or indirectly with the protein moiety to yield a protein-polysaccharide conjugate.

According to another embodiment, the protein is functionalized with at least one pendent amino-oxy group, which is subsequently reacted with a carbonyl group on a polysaccharide, oligosaccharide, carbohydrate, or carbohydrate-containing moiety. The carbonyl group is formed with, for example, sodium periodate. For example, in the case of a polysaccharide, the functionalized protein is reacted with the polysaccharide to form a protein-polysaccharide conjugate. The following scheme illustrates a non-limiting aspect of this process:

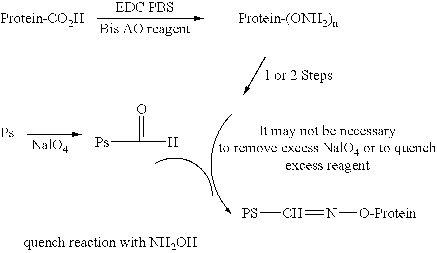

Methods for functionalizing a protein with an amino-oxy group are known to those of ordinary skill in the art. The protein can be functionalized with amino-oxy groups chemically, enzymatically or by genetic engineering. Described herein are methods for functionalizing the protein on either amines or carboxyl groups, and for controlling the number of amino-oxy groups on the protein.

In yet another embodiment, the polysaccharide is functionalized with pendent amino-oxy groups and subsequently reacted with a glycoprotein containing carbonyl groups. These may be present, for example, by oxidizing the carbohydrate on the glycoprotein. Aldehydes may be created by selective oxidation of N-terminal serine or threonine.

In accordance with the present invention, for example when the polysaccharide, oligosaccharide, carbohydrate, or carbohydrate-containing moiety is functionalized with an amino-oxy group, the protein advantageously contains at least one carbonyl group in the form of, e.g., a ketone or aldehyde moiety. Aldehydes may be created on proteins containing an N-terminal serine or threonine, and the resulting protein can be reacted with an amino-oxy reagent, thus uniquely functionalizing the N-terminal. This monovalently-functionalized protein can then be reacted directly, for example, with a carbonyl-containing polysaccharide, if the amino-oxy reagent is homofunctional or indirectly, using spacers. N-terminal serine or threonine can occur naturally, or be engineered into a protein.

In the instances where the protein is functionalized with at least one amino-oxy group, the polysaccharide, oligosaccharide, or carbohydrate contains at least one carbonyl group. The carbonyl groups may be a natural part of the polysaccharide structure, e.g., the reducing end of the polymer, or created, for example, by oxidation. Reductive amination has been widely used to produce protein-polysaccharide conjugates. As a result, means to produce carbonyl-containing polysaccharides are well-known to those versed in the art.

Some polysaccharides contain a reducing sugar on their end, e.g., Hib PRP and Neisseria PsC. These contain aldehydes as hemiacetals and can be reacted with amino-oxy reagents. Additional aldehydes may be created by specific degradation of the polysaccharide. General procedures are described in, for example, Lindberg et al. “Specific Degradation of Polysaccharides—Adv in Carbohydrate Chemistry and Biochemistry,” Tipson et al., eds. Vol 31, pp. 185-240 (Academic Press, 1975). For example, when PRP is oxidized with sodium periodate, the polysaccharide chain is cleaved so as to produce oligosaccharides with an aldehyde on each end.

Many other methods for creating aldehydes are known to those versed in the art. For example Jennings et al., U.S. Pat. No. 4,356,170 entitled “Immunogenic Polysaccharide-Protein Conjugates”; Tai et al., U.S. Pat. No. 5,425,946 entitled “Vaccines against Group C Neisseria Meningitidis”; Porro, U.S. Pat. No. 5,306,492 entitled “Oligosaccharide Conjugate Vaccines”; Yang et al., U.S. Pat. No. 5,681,570 entitled “Immunogenic conjugate molecules”; Constantino et al., “Development and phase 1 clinical testing of a conjugate vaccine against meningococcus A and C,” Vaccine 10: 691 (1992); Laferriere et al., “The synthesis of Streptococcus pneumoniae polysaccharide-tetanus toxoid conjugates and the effect of chain length on immunogenicity,” Vaccine, 15: 179 (1997).

In accordance with the present invention, it may be desirable to add aldehyde moieties to proteins and/or polysaccharides. Those of ordinary skill in the art will appreciate that there are many acceptable methods for doing so. Suitable non-limiting examples of methods to add aldehydes to proteins and polysaccharides include the following:

1. Hydroxyl groups are reacted with chlorohexanol dimethyl acetal in a base, and the masked aldehyde is subsequently revealed by mild acid hydrolysis. Dick et al., Conjugate Vaccines (Eds. Cruse, et al.), pp. 91-93 (1989).

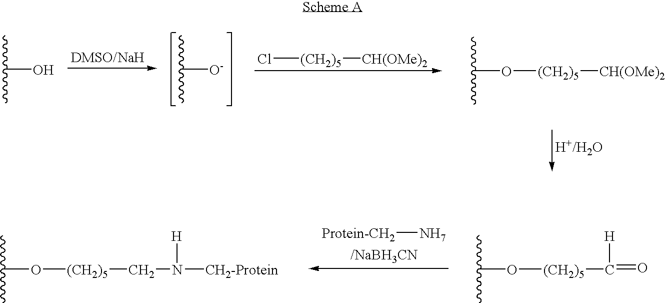

2. Glucouronic lactone and sodium cyanoborohydride are used to reductively aminate protein amines. Saponification is used to open the lactone. The sugar is then oxidized to an aldehydes using sodium periodate.

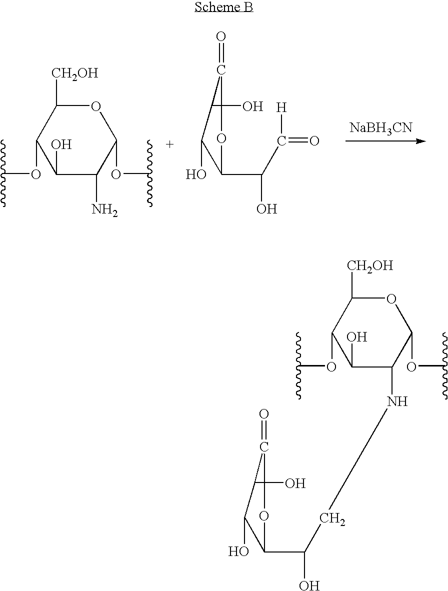

3. A carboxylated carbohydrate, for example, glucuronic acid, galactaric acid, glyceric acid, or tartaric acid is added to protein amines using a carbodiimide reagent. The glycosylated protein is then oxidized to create aldehyde moieties using sodium periodate.

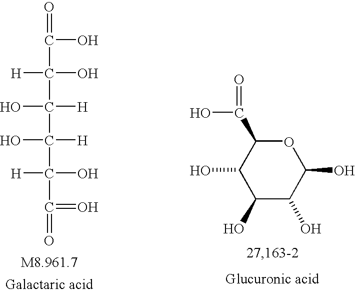

4. Aldehydes can also be created via enzymatic oxidation, using suitable oxidizing enzymes such as, for example, glucose oxidase, galactose oxidase, and neurominidase. For example, neurominidase may be used to remove terminal sialic acid, followed by galactose oxidase. (Hermanson, Bioconjugation Techniques, p. 116-117).

5. Chemical addition of aldehydes to amines on proteins or polysaccharides can be effected using succinimidyl-p-formyl benzoate or succinimidyl-p-formylphenoxyacetate. These NHS esters of aldehydes react with amines and result in the addition of an aldehyde.

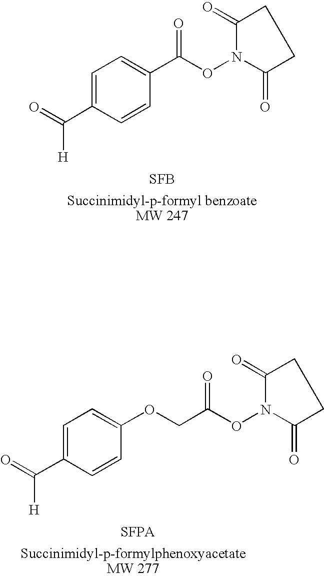

6. Still another method uses the reaction of a bis-aldehyde (e.g., gluteraldehyde) with an amine. (Hermanson, Bioconjugation Techniques, p. 119-120).

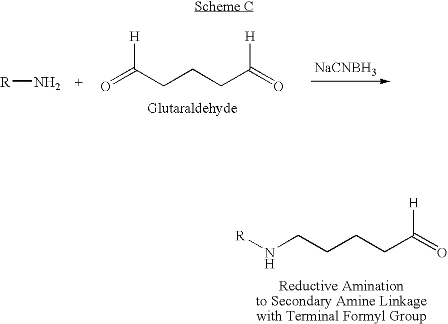

7. Another suitable process is the addition of glyceraldehydes to protein amines using reductive amination, followed by oxidation with sodium periodate to create aldehydes.

Optionally, if the conjugate contains residual free amino-oxy groups or aldehydes, and if it is desired to quench these groups, an additional step may be taken. One of the methods for quenching a conjugate having an aldehyde is by reduction, e.g., using sodium borohydride. Alternatively, residual carbonyls may be quenched with a mono amino-oxy reagent, e.g., amino-oxy-acetate. Residual amino-oxy groups can be quenched with a monofunctionalcarbonyl, e.g., glyceraldehyde, acetone or succinic semialdehyde.

E. Amino-Oxy Reagents

The preparation of conjugate vaccines may be accomplished by the use of various amino-oxy reagents. A variety of useful homofunctional and heterofunctional amino-oxy reagents may be prepared by one skilled in the art, and may also be obtained from Solulink, Inc.™, 9853 Pacific Heights Blvd., Suite H, San Diego, Calif. 92121, and still others are described in the literature. Many more can be conceived of and easily synthesized. Toyokuni et al., “Synthesis of a new heterofunctional linker, N-[4-(amino-oxy)butyl]maleimide for facile access to a thiol-reactive 18F-labeling agent.” Bioconjugate Chem. 14: 1253 (2003).

Suitable non-limiting examples of reagents that may be used in accordance with the present invention include those prepared by Solulink™ (San Diego, Calif.). For example, bis(amino-oxy)cystamine is a homofunctional amino-oxy-reagent that can be converted to a heterofunctional thiol-amino-oxy reagent. “Boc” is the art-recognized acronym for the t-butoxy carbonyl protecting group. Boc-amino-oxy acetate can be used to synthesize a number of suitable amino-oxy reagents according to, for example, the following scheme:

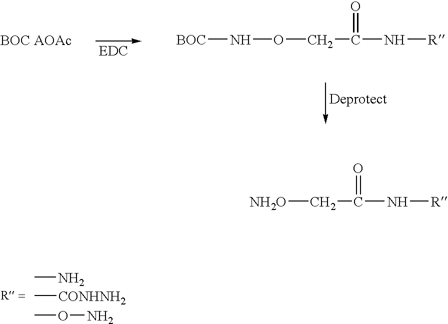

The ligands identified by R″ are suitable, non-limiting examples of nucleophilic ligands that may be used in accordance with the present invention.

The above reagents are based on 2-(Boc-amino-oxy)acetic acid, available from Bachem (Prod. No. A4605.005). Other useful starting reagents for making amino-oxy reagents include N-Boc-hydroxylamine and N-Fmoc-hydroxylamine. These reagents are available from Aldrich Chemical. N-Boc-Hydroxylamine can be used to prepare a useful amino-oxy reagent as follows:

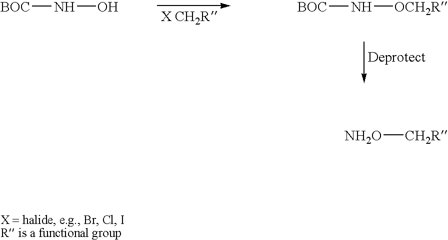

Homofunctional amino-oxy reagents may be used in accordance with the present invention. Suitable homofunctional amino-oxy reagents that may be used include, for example, bis(amino-oxy)ethylene diamine, bis(amino-oxy)butane, and bis(amino-oxy)tetraethylene glycol, all of which are known and can be prepared by art-recognized methods. For example, bis(amino-oxy)butane may be prepared as follows:

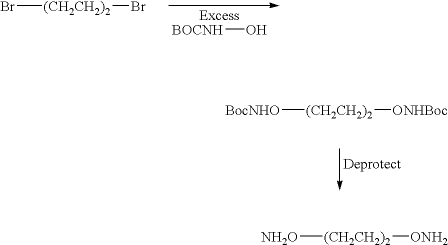

Synthesis of various useful heterofunctional amino-oxy reagents have been described in the literature, for example Mikolajczyk et al., Bioconjugate Chem. 5: 636 (1994) (a maleimide-amino-oxy reagent); Mikola & Hanninen Bioconjugate Chem. 3: 182 (1992) (amino-oxy alklyamines); Webb & Kaneko Bioconjugate Chem. 1: 96 (1990) (amino-oxy-dithionitropyridyl reagents). Jones et al. describe the synthesis of amino-oxy ethers from N-Boc hydroxylamine and alkyl iodides and bromides, which provide another route to useful amino-oxy reagents. Dixon & Weiss, J. Org Chem. 49: 4487 (1984), describe bis-amino-oxy reagents that may be used in accordance with the present invention.

Ketones may be added to amines using, for example, reagents like NHS levulate (from Solulink™). Carbohydrate groups on a protein, e.g., glycoproteins, can be oxidized to carbonyls with, for example, sodium periodate. In addition, reverse proteolysis may be used to add carbonyls or amino-oxy groups as described in Rose et al., “Preparation of well-defined protein conjugates using enzyme-assisted reverse proteolysis,” Bioconjugate Chem. 2: 154 (1991). N-terminal threonines or serines on proteins may be selectively oxidized to aldehydes.

Small linker molecules may also be used to functionalize proteins and polysaccharides with amino-oxy groups. See, for example, Vilaseca et al., “Protein conjugates of defined structure: synthesis and use of a new carrier molecule,” Bioconj. Chem. 4: 515 (1993); and Jones et al., “Synthesis of LJP 993, a multivalent conjugate of the N-terminal domain of b2GPI and suppression of an anti-b2GPI immune response,” Bioconj. Chem. 12: 1012 (2001).

As is known to those of ordinary skill in the art, amino-oxy, aminooxy, aminoxy, and oxy-amine are all synonymous terms.

F. Indirect Conjugation

As stated above, the conjugation between the first moiety and the second moiety may proceed either indirectly or directly. In certain instances, the process of combining a protein and a polysaccharide may lead to undesirable side effects. In some cases, direct coupling can place the protein and the polysaccharide in very close proximity to one another and encourage the formation of excessive crosslinks between the protein and the polysaccharide. Under the extreme of such conditions, the resultant material can become very thick (e.g., in a gelled state).

Over-crosslinking also can result in decreased immunogenicity of the protein and polysaccharide components. In addition, the crosslinking process can result in the introduction of foreign epitopes into the conjugate or can otherwise be detrimental to production of a useful vaccine. The introduction of excessive crosslinks exacerbates this problem.

Control of crosslinking between the protein and the polysaccharide can be controlled by the number of active groups on each, concentration, pH, buffer composition, temperature, the use of spacers and/or charge, and other means well-known to those skilled in the art.

For example, a spacer may be provided between the protein and polysaccharide in order to control the degree of crosslinking. The spacer helps maintain physical separation between the protein and polysaccharide molecules, and it can be used to limit the number of crosslinks between the protein and polysaccharide. As an additional advantage, spacers also can be used to control the structure of the resultant conjugate. If a conjugate does not have the correct structure, problems can result that can adversely affect the immunogenicity of the conjugate material. The speed of coupling, either too fast or too slow, also can affect the overall yield, structure, and immunogenicity of the resulting conjugate product. Schneerson et al., Journal of Experimental Medicine, 152: 361 (1980).

G. Vaccine Compositions

This invention further relates to vaccines and other immunological reagents that can be prepared from the conjugates produced by the method in accordance with the invention. For example, to produce a vaccine or other immunological reagent, the conjugates produced by the method according to the invention may be combined with a pharmaceutically acceptable medium or delivery vehicle by conventional techniques known to those skilled in the art. Such vaccines or immunological reagents will contain an effective therapeutic amount of the conjugate according to the invention, together with a suitable amount of vehicle so as to provide the form for proper administration to the patient. These vaccines may include alum or other adjuvants.

Exemplary pharmaceutically acceptable media or vehicles include, for example, sterile liquids, such as water and oils, including those of petroleum, animal, vegetable or synthetic origin, such as peanut oil, soybean oil, mineral oil, sesame oil, and the like. Saline is a preferred vehicle when the pharmaceutical composition is administered intravenously. Aqueous dextrose and glycerol solutions can be employed as liquid vehicles, particularly for injectable solutions. Suitable pharmaceutical vehicles are well known in the art, such as those described in E. W. Martin, Remington's Pharmaceutical Sciences.

The vaccines that may be prepared in accordance with the invention include, but are not limited, to Diphtheria vaccine; Pertussis (subunit) vaccine; Tetanus vaccine; H. influenzae type b (polyribose phosphate); S. pneumoniae, all serotypes; E. coli, endotoxin or J5 antigen (LPS, Lipid A, and Gentabiose); E. coli, O polysaccharides (serotype specific); Klebsiella, polysaccharides (serotype specific); S. aureus, types 5 and 8 (serotype specific and common protective antigens); S. epidermidis, serotype polysaccharide I, II, and III (and common protective antigens); N. meningitidis, serotype specific or protein antigens; Polio vaccine; Mumps, measles, rubella vaccine; Respiratory syncytial virus; Rabies; Hepatitis A, B, C, and others; Human immunodeficiency virus I and II (GP120, GP41, GP160, p24, others); Herpes simplex types 1 and 2; CMV (cytomegalovirus); EBV (Epstein-Barr virus); Varicella/Zoster; Malaria; Tuberculosis; Candida albicans, other candida; Pneumocystis carinii; Mycoplasma; Influenzae viruses A and B; Adenovirus; Group A streptococcus, Group B streptococcus, serotypes, Ia, Ib, II, and III; Pseudomonas aeroginosa (serotype specific); Rhinovirus; Parainfluenzae (types 1, 2, and 3); Coronaviruses; Salmonella; Shigella; Rotavirus; Enteroviruses; Chlamydia trachomatis and pneumoniae (TWAR); and Cryptococcus neoformans.

The invention also relates to the treatment of a patient by administering an immunostimulatory amount of the vaccine. The term “patient” refers to any subject for whom the treatment may be beneficial and includes mammals, especially humans, horses, cows, pigs, sheep, deer, dogs, and cats, as well as other animals, such as chickens. An “immunostimulatory amount” refers to that amount of vaccine that is able to stimulate the immune response of the patient for prevention, amelioration, or treatment of diseases. The vaccines of the invention may be administered by any suitable route, but they preferably are administered by intravenous, intramuscular, intranasal, or subcutaneous injection. For example, carbohydrate-based vaccines can be used in cancer therapy.

In addition, the vaccines and immunological reagents according to the invention can be administered for any suitable purpose, such as for therapeutic, prophylactic, or diagnostic purposes.

The invention also relates to a method of preparing an immunotherapeutic agent against infections caused by bacteria, viruses, parasites, fungi, or chemicals by immunizing a patient with the vaccine described above so that the donor produces antibodies directed against the vaccine. Antibodies may be isolated or B cells may be obtained to later fuse with myeloma cells to make monoclonal antibodies. The making of monoclonal antibodies is generally known in the art (see Kohler et al., Nature, 256: 495 (1975)). As used herein, “immunotherapeutic agent” refers to a composition of antibodies that are directed against specific immunogens for use in passive treatment of patients. A plasma donor is any subject that is injected with a vaccine for the production of antibodies against the immunogens contained in the vaccine.

### EXAMPLES

### Example 1

### Preparation of an Amino-Oxy Functionalized Protein

The following example illustrates the preparation of an amino-oxy functionalized protein that can be conjugated to a polysaccharide. Bovine serum albumin (BSA) was used as a model protein.

Bis(amino-oxy)tetraethylene glycol was linked to carboxyl groups on bovine serum albumin (BSA) with carbodiimide. Monomer BSA was prepared as described in (Lees et al., Vaccine 14: 190, 1996). Bis(amino-oxy)tetraethylene glycol (85 mg) (prepared by Solulink™, MW 361) was made up in 850 μl of 0.5 M HCl. 5 N NaOH was added to adjust to a pH ˜4.5. 1 ml of BSA mono (42.2 mg/ml in saline) was added. The reaction was initiated by the addition of 25 μl of freshly prepared EDC (1-(3-dimethylamino)propyl)-3-ethylcarbodiimide hydrochloride, 100 mg/ml in water). After approximately 3 hours, the solution was dialyzed overnight against saline at 4° C. The solution was then made up to 4 ml with saline and concentrated with an Amicon Ultra 4™ centrifugal device (30 kDa cutoff) to ˜0.5 ml, and was further desalted on a 1×15 cm G-10 column (Pharmacia) equilibrated with saline. The void volume fraction was then concentrated to ˜1 ml using the Amicon Ultra 4™ device. Using the BCA assay (Pierce Chemical Co), the protein concentration was estimated to be 34 mg/ml BSA. Trinitrobenzene sulfonic acid assay gave an intense red/orange, indicating the presence of amino-oxy group.

### Example 2

### Preparation of an Amino-Oxy Derivatized Polysaccharide

The following example illustrates the preparation of any amino-oxy functionalized polysaccharide that can be conjugated to a protein, peptide, or hapten.

Pn14 (10 ml at 5 mg/ml in water) was activated by the addition of 40 mg of CDAP (100 mg/ml stock in acetonitrile), followed by triethylamine to raise the pH to 9.4. After approximately 2.5 minutes, 4 ml of 0.5 M hexanediamine (pH 9.4) was added. The reaction was permitted to proceed for about 2 hours. Excess reagent was then removed by dialysis against saline to yield amino-Pn14.

Amino-Pn14 was then reacted with excess NHS bromoacetate at pH 8 and dialyzed against saline in the dark at 4° C. The bromoacetylated Pn14 was concentrated by pressure filtration and then dialyzed against water.

Amino-oxy cysteamine was prepared from bis amino-oxy cystamine by TCEP reduction followed by ion exchange on a Dowex 1X-8 column as follows:

Bis(amino-oxy)cystamine (obtained from Solulink) was made up in 50% NMP/water at 0.1 M. TCEP was made up in water at 0.5 M and 3× molar equivalents of 1 M sodium bicarbonate was added. A 1.5 molar excess of TCEP was combined with Bis(AO)cystamine, and adjusted to pH ˜7 with sodium carbonate. After 10 minutes, the mixture was diluted 5-fold into 10 mM bistris at pH 5. The reaction mixture was applied to a 1×3 cm Dowex 1-x8 column that had been washed with 1 M NaCl and equilibrated with 10 mM bistris, pH 5. The reduced amino-oxy cysteamine is found in the flow through of the column.

Amino-oxy cysteamine was added to the bromoacetylated Pn14 and reacted at pH 8 in the dark. The reaction mixture was then concentrated, diafiltered, and then dialyzed against water.

Pn14 concentration was determined to be 9.1 mg/ml by the resorcinol/sulfuric acid method. Using the TNBS assay and amino-oxy acetate as the standard, the amino-oxy concentration was estimated at 0.74 mM, resulting in about 8 amino-oxy groups per 100 kDa of polysaccharide.

### Example 3

### Preparation of a BSA-Dextran Conjugate

The following example illustrates the preparation of a conjugate vaccine using an amino-oxy functionalized protein and an oxidized polysaccharide. Specifically, the amino-oxy functionalized BSA prepared in Example 2 was linked to oxidized dextran.

Dextran was oxidized using sodium periodate as follows: A 10 mg/ml solution of T2000 dextran (Pharmacia) was made to 10 mM in sodium acetate, pH 5 and then 10 mM sodium periodate (from a 0.5 M stock in water), and incubated at room temperature in the dark. At 1, 5, 10 and 15 min, an aliquot was removed, quenched by the addition of glycerol, and dialyzed against water in the dark. The final concentration of dextran was determined to be about 4.5 mg/ml.

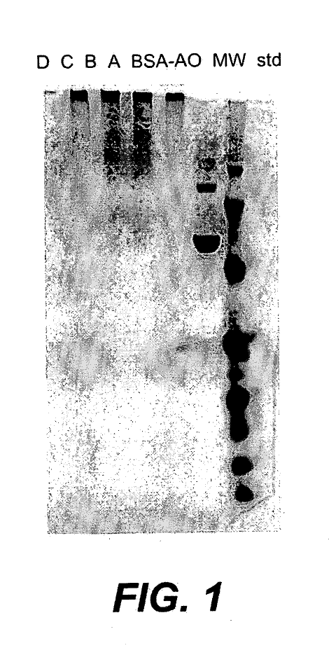

The protein was conjugated to the polysaccharide as follows: 110 μl of each oxidized dextran preparation (1-15 min oxidation) was combined with 15 μl BSA-amino-oxy (0.5 mg each). After an overnight reaction in the dark at room temperature, the samples were analyzed by SDS PAGE (4-12% gradient gel, NuPAGE, Invitrogen). With reference to FIG. 1, lanes are conjugates prepared with (A) dex ox 1 min; (B) dex ox 5 min; (C) dex ox 10 min; (D) dex ox 15 min; BSA-amino-oxy only. It is evident that each of the conjugation reactions resulted in high molecular weight material that did not enter the gel. Essentially no unconjugated protein is evident, indicating a high degree of conjugation.

The four conjugates were pooled & fractionated on a S-400HR™ gel filtration column (1×60 cm), equilibrated with saline. The void volume fractions were pooled and assayed for protein and polysaccharide. It was determined that the pool contained 0.21 mg/ml BSA and 0.27 mg/ml dextran. At least 50% of the initial protein and polysaccharide were recovered. Thus, the amino-oxy-protein with oxidized polysaccharide yielded soluble conjugate in excellent yield.

### Example 4

### Preparation of AO-Functionalized TT

The following hypothetical example illustrates the preparation of Tetanus toxoid derivatized with amino-oxy groups using a two-step method.

1 ml tetanus toxoid (10 mg/ml) in 2 M NaCl is made to pH 8 by the addition of 50 μl 1 M HEPES, pH 8. The protein is bromoacetylated by the addition of 7 μl of 0.1 M NHS bromoacetate. After a 1 hour incubation, 2 pmoles of aminocysteamine is added. After an overnight reaction, excess reagent is removed by dialysis against 2 M NaCl.

The protein concentration is determined using the BCA assay (Pierce Chemical) and the presence of the amino-oxy group confirmed using TNBS.

### Example 5

### Preparation of Amino-Oxy-Derivatized BSA Using a Two-Step Method

Bromoacetylation of BSA:

4.1 ml of monomeric BSA (48.5 mg/ml) was made to pH 8 by the addition of 400 μl 1 M HEPES, pH 8 and 5.5 ml water. 1 ml of 0.2 M NHS bromoacetate (ProChem) in NMP was slowly added while vortexing. After an overnight reaction at room temperature in the dark, the solution was dialyzed against saline for 2 days, centrifuged and filtered. 10.6 ml of BSA at 15.3 mg/ml was obtained.

Preparation of Amino-Oxy Cysteamine:

51.5 mg of Bisaminoxocystamine was added to a solution of 56 mg TCEP made up in 1.1 ml 1 M sodium carbonate, 586 μl DMSO, and 586 μl water. After 15 minutes, the TCEP was removed on a 1×5 cm Dowex 1x-8 column, equilibrated with 10 mM Bistris, pH 6. The DTNB positive flow thru was pooled and found to be 22.6 mM thiol.

6 ml was added to the bromoacetylated BSA and the pH adjusted to 8. The reaction was allowed to proceed overnight in the dark, and was then dialyzed for 2 days at 4° C. against multiple changes of saline. The amino-oxy BSA was determined to be about 8.6 mg/ml. Reaction of an aliquot with TNBS at pH 8 gave an orangish color, indicating the presence of the amino-oxy group.

### Example 6

### Use of CDAP to Prepare Amino-Oxy Derivatized Polysaccharide and Amino-oxy Conjugates

This experiment illustrates the use of CDAP to prepare amino-oxy derivatized polysaccharide and amino-oxy conjugates. It illustrates how chemistry other than oxidation can be used to functionalize a polysaccharide with amino-oxy groups.

I. Preparation of an Amino-Oxy Derivatized Polysaccharide Using CDAP Chemistry

A solution of bifunctional amino-oxy reagent was prepared by solubilizing 29 mg of bis-amino-oxy acetate (ethylene diamine) (prepared by Solulink™) in 200 μlμM NaAc, pH 5. Dextran was activated using CDAP chemistry as follows. To a solution of 0.5 ml T2000 dextran at 10 mg/ml in water, 25 μl of CDAP (100 mg/ml acetonitrile) was added and 30 seconds later the pH was raised by the addition of 25 μl 0.2 M triethylamine (TEA) and three 5 μl of TEA neat.

At 2.5 minutes, the pH was reduced by the addition of 100 μl 1 M NaAc, pH 5. 200 μl of the BisAO solution was then added. After ˜30 minutes reaction, the solution was desalted on a 1×15 cm P6DG column (BioRad) equilibrated with NaAc buffer (10 mM NaAc, 150 mM NaCl, 5 mM EDTA, pH 5). The desalted polysaccharide was estimated at 1.7 mg/ml dextran, using the resorcinol assay, and about 11 amino-oxy groups/100 kDa dex using a TNBS assay.

II. Preparation of Oxidized Ovalbumin

To a 0.4 ml solution of ovalbumin (14.4 mg) (OVA), 10 μl of 1 M sodium acetate, pH 5 was added, followed by the addition of 10 μl 0.5 M sodium periodate (in water). After a 15 minutes incubation at room temperature in the dark, the reaction was quenched with the addition of a few drops of 50% glycerol. The reaction mixture was then dialyzed in the dark against NaAc buffer. By adsorption at 280 nm, the concentration of oxidized ovalbumin (“OVA(ox)”) was 6.6 mg/ml.

III. Preparation of Conjugates and Controls

The following solutions were prepared and each was incubated overnight at room temperature in the dark:
A. 500 μl Dex AO (0.85 mg)+75 μl OVA(ox)+100 μl 1 M NaAc pH 5.
B. 250 μl Dex AO (0.0.43 mg)+37.5 μl NaAc buffer+50 μl 1 M NaAc
C. 250 μl NaAc buffer+37.5 μl OVA(ox)+50 μl 1 M NaAc.

Each was then assayed by SDS PAGE and SEC HPLC. Only sample A contained high molecular weight (HMW) material, with ˜20% of protein conjugated, as estimated by SEC HPLC. Neither B nor C indicated any HMW material by SEC HPLC or SDS PAGE.

### Example 7

### Use of Cyanogen Bromide to Label Polysaccharide with a Bis-Amino-Oxy Reagent

This prophetic example demonstrates the derivatization of a polysaccharide with an amino-oxy reagent using cyanogen bromide (CNBr).

Polysaccharide (e.g., Pn-14) is made up at 10 mg/ml in water, and is treated with CNBr at 1 mg per mg of polysaccharide at pH 10.5 for 6 minutes in a pH-stat. The reaction mixture is then reduced to ˜pH 7 by the addition of 0.5 M bis-amino-oxy reagent (e.g., bis-AO(EDA). After an overnight reaction, the solution is dialyzed into water and assayed for amino-oxy groups with TNBS, and for carbohydrates with the resorcinol assay. This amino-oxy derivatized polysaccharide is used for conjugation with a carbonyl-containing protein.

According to another embodiment, the CNBr-activated polysaccharide can be reacted with amino-oxy acetate. This will result in a polysaccharide functionalized with carboxyl groups. The carboxyl groups can then be further functionalized and indirectly or directly linked to proteins (with, for example, carbodiimide).

### Example 8

### Conjugation of Amino-Oxy Derivatized Protein with Oxidized Polysaccharide

This example illustrates the preparation of amino-oxy derivatized protein with the functionalization occuring on the amines. This amino-oxy derivatized protein is then covalently linked to the clinically relevant polysaccharides Neisseria meningididis A and C.

I. Functionalization of Protein with Amino-Oxy Groups of a Protein on its Amines (Protein with Pendent Amino-Oxy Groups on Amines)

Amines on the protein are bromoacetylated and then reacted with a thiol-amino-oxy reagent to produce a protein with pendent amino-oxy groups. Bis(amino-oxy acetate)cystamine 2HCl was prepared by Solulink.™ Monomeric BSA was at 42.2 mg/ml. NHS bromoacetate was obtained from Prochem and made up at 0.1 M in NMP (N-methyl-2-pyrrolidone). The amino-oxy protein was prepared as follows. In each of 2 tubes, a solution of 0.5 ml of BSA (21.1 mg) and 250 μl H₂O+100 μl 1 M HEPES, pH 8 was prepared. One tube was reacted with a 30 fold molar excess of NHS bromoacetate (93 μl) and the other at a 10 fold molar excess (31 μl).

After about 1 hr, each was made up to 15 ml with sodium acetate buffer (10 mM NaAc, 0.15 M NaCl, 5 mM EDTA, pH 5) and concentrated to about 200 μl using an Amicon Ultra 15™ device (30 kDa cutoff).

Amino-oxy acetate cysteamine was prepared as follows:

To a solution of 9.8 mg of Bis(AOAc)cystamine (prepared by Solulink™) in 114 μl 1 M sodium acetate+114 μl NMP, 22.8 μl of 0.25 M TCEP in 1 M HEPES, pH 8 was added as a reducing agent. After 1 hour, the partially reduced amino-oxy thiol reagent was added to each of the bromoacetylated BSA preparations, the pH was adjusted to about pH 8 and the reaction allowed to proceed overnight in the dark at 4° C.

Each was desalted using the Amicon Ultra 15™ device by making volume up to 15 ml with NaAc buffer and centrifuging. The desalting process was repeated four times. The final volume was about 200 μl and was then made up to about 1 ml with NaAc buffer. This product was termed BSA-S-AO. By adsorbance at 280 nm, the 30× prep was determined to be 29.8 mg/ml and the 10× prep 24.8 mg/ml.

II. Preparation of Oxidized Neisseria meningiditis Polysaccharide A and C (Neiss PsA and Neiss PsC)

Neiss PsA and PsC were solubilized overnight at room temperature at 10 mg/ml in water and then stored at 4° C. 50 μl of 1 M sodium acetate, pH 5, was added to 1 ml of each polysaccharide solution, followed by the addition of 25 μl 0.5 M sodium periodate (0.5 M in water). After 10 minutes in the dark at room temperature, each was dialyzed 4 hours against 4 l water. Each was then made up to 4 ml with water and further desalted using an Amicon Ultra 4™ device (30 kDa cutoff). Using the resorcinol assay, the oxidized Neiss PsA was determined to be 12.1 mg/ml and the oxidized Neiss PsC was 17.8 mg/ml.

III. Conjugation of BSA-S-AO with Oxidized Neiss PsA and PsC

The following mixtures of BSA-S-AO and oxidized PsA and PsC were prepared.

| Conjugate | μl Ps | μl BSA | 1 M NaAc pH 5 |
|---|---|---|---|
| BSA10x-PsA | 175 μl (2.1 mg) | 134 μl (4 mg) | 25 μl |
| BSA30x-PsA | 175 μl (2.1 mg) | 161 μl (4 mg) | 25 μl |
| BSA10x-PsC | 175 μl (3.1 mg) | 134 μl (4 mg) | 25 μl |
| BSA30x-PsC | 175 μl (3.1 mg) | 161 μl (4 mg) | 25 μl |

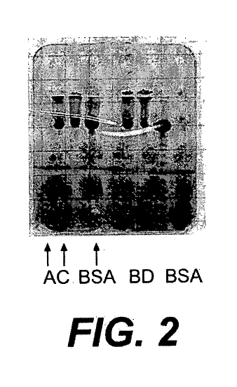

After an overnight reaction at room temperature in the dark, conjugates were assayed by SDS PAGE using a Phast gel (8-25%)(Pharmacia) under reducing conditions. With reference to FIG. 2, from left to right the lanes are BSA30x-PsA, BSA30x-PsC, BSA30x, BSA10x-PsA, BSA10x-PsC, BSA10x. It is seen that there is a significant amount of high molecular weight materials that did not enter the gel, indicating that conjugation of the protein to the polysaccharide occurred.

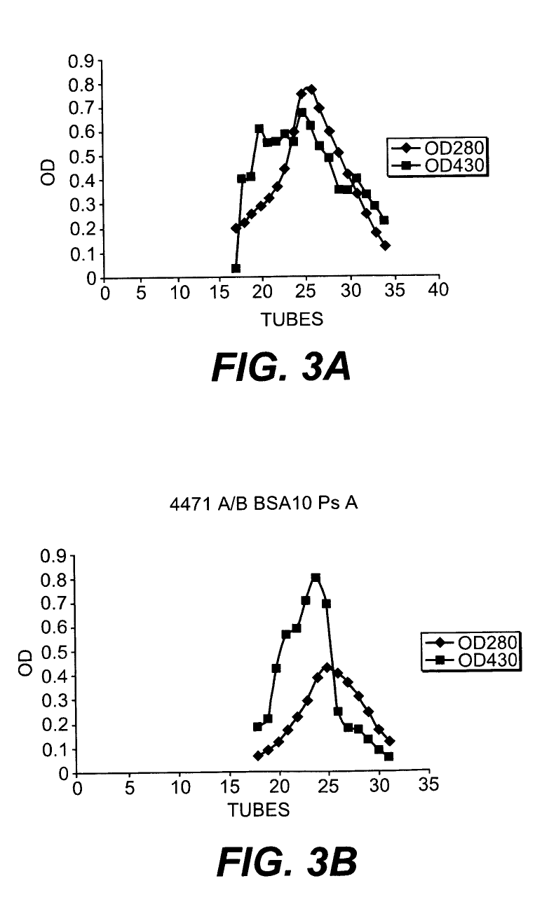

The PsA conjugates were pooled and fractionated by gel filtration on a S-400HR column (1×60 cm, Pharmacia), equilibrated with saline. Similarly, the PsC conjugates were pooled and fractionated. Approximately 1 ml fractions were collected and assayed for protein (by absorbance) and for carbohydrate using the resorcinol assay. The results are provided in FIG. 3.

For the PsC conjugate, tubes 18-22 were pooled and for the PsA conjugate, tubes 19-23 were pooled and examined by SDS PAGE using reducing conditions.

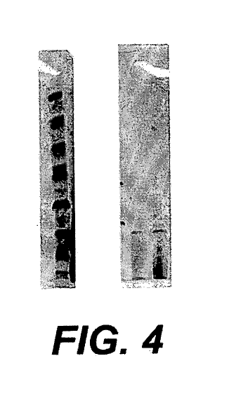

With reference to FIG. 4, the BSA-Neiss PsC conjugate is on the left and the PsA conjugate is next to it. On the right is the molecular weight standard. A small amount of free BSA is observed in each, indicating incomplete separation of the conjugated and free protein. Each contains a significant amount of conjugated high molecular weight material that did enter the gel.

### Example 9

### Preparation of (BSA-Levulate)-Amino-Oxy-Pn14 Conjugate

This example illustrates the reaction of an amino-oxy group with a ketone and shows that this can be used for the formation of conjugates and, more specifically, the preparation of (BSA-Levulate)-Amino-oxy Pn14.

NHS Levulate was obtained from Solulink and made up by solubilizing 5.1 mg in 100 μl NMP. This was slowly added to a vortexed solution of 200 μl BSA at 48.5 mg/ml, 200 μl water, and 100 μl 1 M HEPES, pH 8. After an overnight reaction, the mixture was diafiltered using an Amicon Ultra 15 device, (30 kDa cutoff). The final volume was 0.5 ml. This product is BSA-LEV.

100 μl of BSA-LEV was combined with 300 μl of amino-oxy Pn14 (4.5 mg/ml Pn14) and incubated for several days in the dark. The conjugate and the individual components were assayed by SEC HPLC using a Superose 6 column (Pharmacia). The conjugate was then fractionated on an S400HR column. Protein was assayed using the Bradford dye method, and polysaccharide with the resorcinol method. The high molecular weight fraction was found to contain 0.6 mg BSA/mg Pn14.

### Example 10

### Preparation of Amino-Oxy-BSA—Neisseria PsC Conjugate

Neiss PsC was oxidized to create terminal aldehyde as generally described in Jennings & Lugowski J. Imm. 127: 1011 (1981). SEC HPLC indicated the molecular weight of the PsC was significantly reduced.

After overnight conjugation of PsC and BSA-AO, analysis was conducted via SEC HPLC Superose 6 0.5 ml/min. The conjugate was fractionated on a 1×60 cm S200HR column, equilibrated 10 mM sodium acetate, 150 mM NaCl, 2 mM EDTA, pH 5. It was determined by both SEC analysis and gel filtration that most of BSA was conjugated. The high molecular weight peak was analyzed for protein and carbohydrate and determined to contain 0.2 mg BSA/mg PsC.

### Example 11

### Preparation of Amino-Oxy-BSA—Neisseria PsA Conjugate

This example illustrates the preparation of a Neisseria PsA-BSA conjugate by way of functionalizing the protein with an amino-oxy group.

Neiss PsA was terminally reduced to an alditol with NaBH₄ and then oxidized to create terminal aldehyde as generally described in Jennings & Lugowski J. Imm. 127: 1011 (1981).

Neisseria PsA was solubilized in water at 20 mg/ml for 15 min. To 1 ml of the solubilized polysaccharide, 10 mg of sodium borohydride was added. The pH was maintained to about 8-9. After 1 hour, 100 μl of 1 M NaAc was added, and the pH was adjusted to 5. The reduced PsA was desalted on a 1×15 cm G10 column, equilibrated with saline, and the void volume fraction concentrated with an Amicon Ultra 4 (10 kDa cutoff device) to about 1 ml. 20 mg of solid sodium periodate was added, along with 100 μl 1 M sodium acetate at pH 5. After a 15 minute oxidation in the dark at room temperature, the reaction was quenched by the addition of a drop of glycerol and then desalted on 1×15 cm G10 column equilibrated with 10 mM NaAC, 150 mM NaCl and 2 mM EDTA, pH 5 (acetate buffer). The void volume was pooled and found to be positive in the BCA assay, indicating the presence of reducing sugar. The material was diafiltered and concentrated with an Ultra 4 device into acetate buffer.

Both the amino-oxy BSA and the PsA(red/ox) were examined by SEC HPLC. The molecular weight of the PsA was markedly reduced by the reduction/oxidation process.

Conjugation

In the conjugation step, 150 μl amino-oxy-BSA at 6 mg/ml was combined with 50 μl PsA (red/ox) and 25 μl 1 M NaAc at pH 5.

After an overnight incubation in the dark at 4° C., the conjugate was analyzed by SEC HPLC (Superose6, saline, 0.5 ml/min). It was seen that the PsA contributed very little absorbance and the AO-BSA increased in molecular weight on conjugation.

The conjugate was fractionated on a 1×60 cm S200HR gel filtration column and the high molecular weight fraction assayed for protein and PsA and was found to contain 0.4 mg BSA/mg PsA.

Conclusion: The reduction/oxidation method works well to create aldehydes that can be linked to amino-oxy-protein. PsA was probably hydrolyzed during the NaBH₄ step, which is at elevated pH.

### Example 12

### Preparation of PRP(ox)-BSA-AO Conjugate

Oxidation of PRP Hib

22.7 mg PRP Hib was made up at 10 mg/ml in water, and combined with 100 μl M NaAc and 46 μl 0.5 M sodium periodate. The reaction proceeded in the dark and on ice for 15 minutes, and was then quenched with 50% glycerol. The reaction mixture was diafiltered into water with an Amicon Ultra 4 (10 kDa cutoff) device, 4×4 ml, final volume was approximately 1 ml. A resorcinol assay was conducted at 10 mg/ml. The sample was positive in the BCA assay, indicating the presence of aldehyde.

2. Conjugation Amino-Oxy BSA

AO-S-BSA was provided at 15 mg/ml. 667 μl BSA-S-AO 10 mg was combined with 100 μl 1 M sodium acetate, at pH 5, and approximately 1 ml PRP(ox), and the reaction was permitted to proceed overnight in the dark. It was then quenched by the addition of 50 μl 0.25 M amino-oxy acetate.

3. Assay by SEC Superose6 Prep Grade HR10/30, Equilibrated PBS 0.5 ml/min, OD 220.

Here, 0.5 ml conjugate was fractionated on 1×60 cm S200HR, and equilibrated PBS. All fractions eluted before BSA, indicating higher MW.

| Fraction | BSA/mg Hib PRP (mg) |
|---|---|
| 9 | 0.7 |
| 10 | 0.6 |
| 11 | 0.4 |
| 12 | 0.5 |

### Example 13

### Preparation of BSA-Pn14 Conjugate via Oxidation of Glycidic Acid

This example illustrates a protocol whereby glycidic acid was added to amines on BSA using carbodiimide. The glycidic acid on the protein was then oxidized and reacted with amino-oxy-Pn14.

I. BSA-Glycidic Acid

Monomeric BSA and glycidic acid (obtained from Fluka Chemical) were combined to a final concentration in water of 12.5 mg/ml and 28 mg/ml, respectively. The pH was adjusted to about 5 and 220 μl of 100 mg/ml EDC in water was added. The pH is kept at about 5 for approximately 1.5 hours, and the reaction was quenched by the addition of 0.025 ml 1 M sodium acetate at pH 5. The reaction mixture is then dialyzed against saline at 4° C. overnight.

II. Oxidation of BSA-Glycidic Acid

100 μl of 1 M sodium acetate at pH 5 was added to 1 ml of BSA-glycidic acid (7.8 mg/ml), followed by 25 μl of 0.5 M sodium periodate in water. After 10 minutes in the dark, glycerol was added to quench the reaction and excess reagent removed using an Amicon Ultra centrifugal device with a 30 kDa cutoff. The final volume was about 400 μl.

100 μl of the oxidized BSA-glycidic acid was combined with 250 μl of amino-oxy Pn14 at 9.3 mg/ml along with 50 μl 1 M sodium acetate at pH 5. An aliquot was evaluated by SEC HPLC (Superose 6 0.5 ml/min, PBS). After an overnight reaction, another aliquot was assayed in the same way. It was seen that a significant portion of the absorbance eluted at the void volume (−15 minutes), indicating that the protein was linked to the high molecular weight Pn14.

Following gel filtration on an S400HR column (Pharmacia), the high molecular weight fraction was determined to contain 0.3 mg BSA/mg Pn14. This ratio is similar to that determined from the percentage of conjugated high molecular weight protein in the above chromatogram.

Thus, the method of linking glycidic acid to protein using carbodiimide provides a way to create aldehydes on proteins that can be subsequently linked to amino-oxy groups.

### Example 14

### Use of Amino-Oxy Chemistry to Link BSA to Dextran

This example illustrates the coupling of an oligosaccharide via its reducing end to amino-oxy derivatized protein.

T40 dextran was made up at 100 mg/ml in water. The number of reducing ends was estimated using the BCA assay with glucose as the standard. It was found that there were 3.5 mM reducing ends/100 mg/ml T40 dextran, so the average molecular weight was taken to be approximately 28,000 kDa

5 mg amino-oxy BSA containing ˜8 amino-oxy/BSA was combined with 2 ratios of T40 dextran at pH 5.

(A) 830 μl BSA-AO 15.3 mg/ml was combined with 620 μl T40 dextran at 100 mg/ml and 100 μl 1 M NaAc at pH 5.

(B) 830 μl BSA-AO 15.3 mg/ml was combined with 3.1 ml T40 dextran at 100 mg/ml and 500 μl 1 M NaAc at pH 5.

Solutions were reacted at room temperature in the dark for 1 week, and then assayed by SEC HPLC.

Both conjugates eluted much earlier than BSA-AO, indicating that their molecular weight has increased. Conjugates were then fractionated by anion ion exchange (IEX). Consistent with the SEC profile, the higher the molecular weight, the lower ionic strength the conjugate eluted. IEX elution fractions were analyzed for the ratio of carbohydrate to protein and plotted on both a weight and mole ratio (using 28 kDa MW for the T40 dextran).

The peak fraction from each IEX elution was analyzed by SEC HPLC (Superose 6 1 ml/min). The absence of monomeric BSA and the increasing MW for high vs. low ratio T40 dex/BSA conjugates. SDS PAGE confirmed the high molecular weight nature of the conjugate IEX eluants.

### Example 15

### Use of Amino-Oxy Chemistry to Link Oligosaccharide and Protein

This example demonstrates the use of amino-oxy chemistry to link an oligosaccharide indirectly via its reducing end to a protein. A general description of the protocol is as follows. The reducing end of T40 dextran (˜40 kDa MW) was reacted with the amino-oxy group of amino-oxy acetate to create a dextran with a single carboxyl group on one end. This carboxy group was then converted to an amine by reaction with ethylenediamine and carbodiimide. The amine-tipped dextran was then thiolated and reacted with maleimide-derivatized BSA, to create a conjugate consisting of a protein with “threads” of carbohydrate extending from it.

I. Addition of Amino-Oxy Acetate to the Reducing End of Dextran

850 mg of T40 dextran (Pharmacia) was solubilized in 850 μl of water overnight at room temperature.

235 mg of amino-oxy acetate was solubilized in a mixture of 850 μl DMSO and 500 μl 1 M sodium acetate, pH 5 and combined with the T40 dextran solution. An additional 500 μl of DMSO was added to make the solution approximately 50% DMSO. After an incubation at about 68° C. for about 6 hours, the solution was extensively dialyzed against water. The product was dextran containing a single carboxyl group on its reducing end.

1.8 g of ethylenediamine 2HCl was added to the solution (approximately 22 ml) and the pH adjusted to approximately 5 with 1 N NaOH. 220 mg of EDC (1-(3-dimethylamino)propyl)-3-ethylcarbodiimide hydrochloride) was added and the pH maintained at about 5 for 3 hours. The reaction was then quenched by the addition of 1 M sodium acetate, pH 5, dialyzed against saline, and concentrated using an Amicon Ultra 15™ (10 kDa cutoff). It was then further dialyzed against saline and then against water.

The product was assayed for amines using TNBS and for carbohydrate using the resorcinol assay. It was determined that there were approximately 0.45 amines per 40,000 kDa of dextran. This product was dextran containing a single amine group on its reducing end and was termed NH₂-AOAc-T40 dextran. Using the resorcinol assay, the solution was determined to have a concentration of about 119 mg/ml dextran. T40 dextran consists of a distribution of molecular weights, which makes it difficult to determine the actual degree of substitution of the reducing ends of the polymers.

II. Thiolated dextran and maleimide-BSA

Maleimide-derivatized BSA was prepared as follows: GMBS (40 μl of a 0.1 M stock in NMP) was added to a solution of 200 μl of monomeric BSA (42.2 mg/ml), 50 μl 0.75 M HEPES, 5 mM EDTA at pH 7.3, and 100 μl water. After a 2 hour reaction, the pH was reduced by the addition of 100 μl 1 M sodium acetate, at pH 5. The solution was desalted using an Amicon Ultra 4™ (30 kDa cutoff) ultrafiltration device and 10 mM NaAc, 0.15 M NaCl, 5 mM EDTA, pH 5.

The NH₂-AOAc-T40 dextran was thiolated using SPDP as follows: 0.5 ml of the NH₂-AOAc-T40 dextran was combined with 100 μl of 1 M HEPES, pH 8 and 100 μl of 0.1 M SPDP were added. After approximately 2 hours, 50 μl of 0.1 M EDTA pH 5 was added, followed by 100 μl of 1 M sodium acetate, pH 5 and 50 μl of 0.5 M dithiothreitol in water. After a 1 hour incubation, the solution was dialyzed into sodium acetate buffer overnight at 4° C.

The thiol tipped T40 dextran and the maleimide derivatized BSA were combined (a small aliquot of the BSA-maleimide was saved for analysis). After an overnight reaction, one half the mixture (about 1 ml) was fractionated by gel filtration using a 1×60 cm S-400HR column, equilibrated with saline. For comparison, a mixture of 100 μl BSA monomer (42.2 mg/ml), 300 μl T40 dextran AOAc, and 0.5 ml saline was similarly fractionated on the same gel filtration column. Fractions (about 1 ml) were analyzed for protein by absorbance at 280 nm and for dextran using the resorcinol assay.

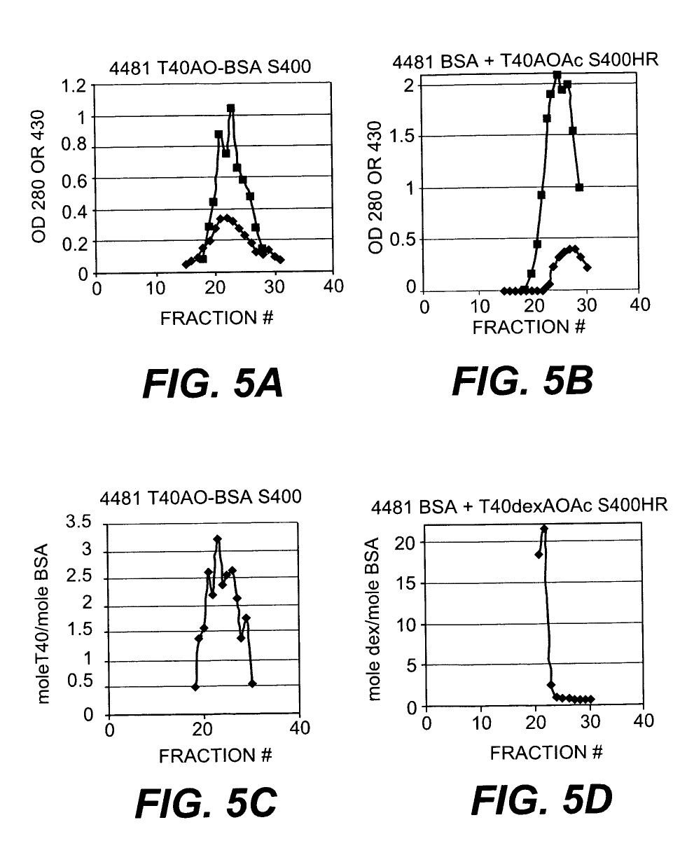

With reference to FIGS. 5A-D, it is evident that the protein and dextran are eluting earlier from the column when in the T40 dextran AOAc-thiol-maleimide BSA conjugate than when the components are mixed. This indicates that a conjugate of higher molecular weight has been formed. Furthermore, the ratio of dextran to protein increased.

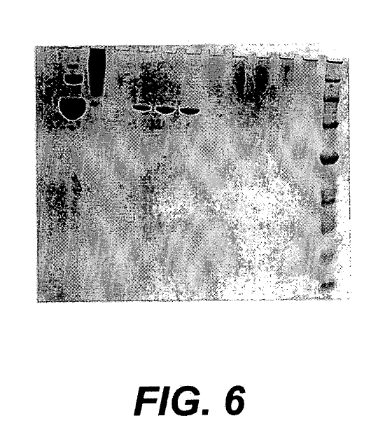

The column fractions were further analyzed by SDS PAGE, with the results provided in FIG. 6. From left to right, MW marker, conjugate fractions 18, 20,22,24,26, mixture fractions #24,26,28, 30, unfractionated conjugate, starting BSA-maleimide.

It is evident that the unfractionated conjugate contains only a small proportion of free protein, indicating that the conjugate was formed in high yield. No high molecular weight protein is evident in the mixture fractions. Only conjugate and essentially no free protein is evident in the conjugate fractions. This confirms that a conjugate formed in high yield.

### Example 16

### Preparation of BSA-Pn14 Conjugates via Glycidic Acid and Amino-Oxy Derivatized Pn-14

The following example is illustrative of the preparation of a conjugate using an aldehyde-substituted protein.

I. In Situ Synthesis of NHS Ester of Glycidic Acid Using TSTU and Addition to BSA

In this step, 7.9 mg of glycidic acid hemi-calcium salt monohydrate (MW 143) was solubilized in 110 μl NMP. This was combined with 200 μl of 0.5 M TSTU (Novachem) in NMP, and 100 μl of triethylamine, and was added to 1 ml of 24 mg/ml BSA. The pH was adjusted to pH 8. After approximately 2 hours, the mixture was dialyzed on 2×1 liter saline. The number of free amines on BSA was determined using TNBS. For the control, the number was 33.2 NH₂/BSA. For glycidic acid/TSTU/BSA, the number was 25 NH₂/BSA. These results lead to the conclusion that BSA was labeled with about 8 glycidic acid units/BSA.

II. Oxidation of Functionalized BSA

A 5 mg aliquot was made up with 25 mM NaAc at pH 5, and 25 mM sodium periodate. The reaction was allowed to proceed in the dark at room temperature for 15 minutes, after which a drop of glycerol was added to quench the reaction. The mixture was fractionated S200HR, pool main peak & concentrate.

III. Preparation of BSA(ox)-AO-Pn14 Conjugate

In this step, 444 μl of amino-oxy functionalized Pn14 was combined with 0.4 ml BSA(ox) made up at 10.1 mg/ml, and 100 μl 1 M NaAc at pH 5, and reacted overnight at room temperature. The Fractionate by gel filtration S400HR 1×60 cm saline+0.02% azide SEC HPLC indicated that oxidation of the Glycdic acid/TSTU/BSA caused polymerization of the BSA.

Also observed was the progressive increase in the high molecular weight peak, indicating that conjugation was increasing with time. The AO-Pn14 alone had minimal absorbance.

### Example 17

### Preparation of Mercaptoglycerol-Bromoacetate BSA

This example illustrates a process for preparing a BSA(mercaptoglycerol(ox))-AO-dextran conjugate.

Preparation of Bromoacetylated BSA

500 μl monomeric BSA (48 mg/ml) was combined with 500 μl 1 M HEPES, at pH 8, and 25 μl 0.1 M NHS bromoacetate in NMP. For the control, 250 μl BSA was combined with 250 μl HEPES and 12 μl NMP

After approximately 1 hour, each was desalted into saline using an Amican Ultra 4 (30 kDa cutoff) device. The final volume was 450 μl, BSA-bromoAc, and 300 μl, BSA control.

Next, 50 mM mercaptoethanol and 50 mM mercaptoglycerol were prepared in water.

Preparation E: 225 μl BSA-BromoAc was combined with 100 μl 1 M HEPES at pH 8 and 50 μl of 50 mM mercaptoglycerol.

Preparation F: The BSA control was combined with 100 μl 1 M HEPES to pH 8 and 50 μl 50 mM mercaptoglycerol.

Preparation G: 225 μl BSA-BromoAc was combined with 100 μl 1 M HEPES at pH 8, and 50 μl 50 mM mercaptoethanol.

After 30 minutes, each was desalted with Amicon Ultra using NaAc buffer (10 mM NaAcetate, 150 mM NaCl, 5 mM EDTA, pH 5). Final volume was 0.5 ml.

Each was then made up in 10 mM sodium periodate from a freshly prepared 0.5 M stock and incubated for 10 minutes at 4° C. in the dark, and then quenched by the addition of glycerol and desalted using the Amicon Ultra device and washed into NaAc buffer. By OD 280, each was determined to be about 20 mg/ml BSA.

Preparation E should contain BSA-aldehyde; Preparation F was not labeled with the bromoacetate, and so it could not react with the mercaptoglycerol. Thus, it should not contain aldehydes. Preparation G would have pendent mercaptoethanol, which does not oxidize, so it should not contain aldehdydes.

315 μl of Amino-oxy dextran, at 15.9 mg/ml, was combined with 250 μl of each BSA preparation, and incubated overnight at room temperature in the dark.

Each was then fractionated by gel filtration on a S400HR 1×60 cm equilibrated with saline. The high molecular weight fraction was analyzed for protein and dextran.

The results indicated that only BSA containing the oxidized mercaptoglycerol formed a conjugate, and this was confirmed by the protein/dextran ratio of the high molecular weight fraction.

| | |
|---|---|
| E BSA-mercaptoglycerol(ox) + AO-dex | 0.97 mg BSA/mg dex. |
| F BSA control(ox) + AO-dex | 0.1 mg BSA/mg dex. |
| G BSA-mercaptoethanol(ox) + AO-dex | 0.1 mg BSA/mg dex. |

### Example 18

### Linking of a Protein to a Polysaccharide via Oxime Formation

The following example illustrates the linking of a protein via its N-terminal group to a polysaccharide via oxime formation.

N-terminal threonine of lysostaphin was oxidized and derivatized with a bis-amino-oxy reagent. Oxidation of the protein was performed as generally described in Gaertner & Offord, “Site-specific attachment of functionalized poly(ethylene glycol) to the amino terminus of proteins,” Bioconjugate Chem. 7: 38 (1996). Lysostaphin a 27 kDa protein was produced in lactococcus.

Trial #1

The lysostaphin used contained only about 30% free N-terminal threonine. Conditions of Gaertner & Offord were used for oxidation of the N-terminal threonine. In more detail, a 50 molar excess of methionine (17.5 μl from a 1M stock in water) was added to 1 ml of a 10 mg/ml solution of lysostaphin. Sodium bicarbonate (1 M) was added to adjust the pH to 8.3. Oxidation was commenced by the addition of sodium periodate (7 μl from a 0.5 M stock in water). The reaction mixture was kept in the dark at room temperature for 10 minutes, at which time 7.1 mg Bis(amino-oxy)tetraethylene glycol (obtained from Solulink™) prepared as a 50 mg/ml solution in DMSO was added. After 1 hour in the dark, the solution was dialyzed against saline in the dark at room temperature. The product is termed lysotaphin AO. The lysostaphin concentration was determined at OD 280 using 0.49 mg/ml/Absorbance unit.

An aliquot was tested with TNBS at pH 5. It has previously been found that amino-oxy but not amines reacted with TNBS under these conditions. The assay was performed as follows: 50 μl of lysostaphin or lysostaphin AO was added to 440 μl of 0.1 M NaAc, pH 5 and then 10 μl of 10 mg/ml TNBS in water added. 5 μl of 1 mM Amino-oxy acetic acid was used as a standard in the above solution. Samples were read at 500 nm after a 6 hour incubation in the dark. The sample solution was orange, indicating presence of amino-oxy groups. Using the standard, it was estimated about 30% of lysostaphin was derivatized with the AO group. This lysostaphin AO was then reacted with excess oxidized T2000 dextran in the dark at room temperature to allow conjugation via oxime formation. The reaction was assayed by SEC HPLC to determine the shift of mass from low molecular weight (unconjugated protein) to high molecular weight (lysostaphin-dextran conjugate). A Phenomenex Biosep SEC2000 (300×4.6) equilibrated with PBS and run at 0.5 ml/min with monitoring at 280 nm was used for SEC HPLC

With reference to FIG. 6, the upper chromatogram is the reaction mixture at about 1 minute, the middle chromatogram is after an overnight reaction and the lower figure is the lysostaphin AO alone. Note the shift to high molecular weight material after the reaction was allowed to proceed overnight. This figure suggests that the AO group on the lysostaphin linked to the high molecular weight, oxidized dextran. About 27% was coupled, based on the percentage of the area of the high molecular weight peak. This is in the expected percentage since only about a third of the lysostaphin contained a free threonine and was derivatized with AO, as indicated by TNBS assay.

### Example 19

### Preparation of DT(ox)-AO-Pn14 Conjugate

This example illustrates the preparation of the DT(ox)-AO-Pn14 conjugate, and it also demonstrates how reagents can be prepared in as “single pot” reactions (which may simplify preparation).

I. Mercaptoglycerol-Diptheria Toxoid

0.5 ml diphtheria toxoid (˜13 mg/ml) was combined with 100 μl 1 M HEPES, pH 8 and 10 μl 0.1 M NHS bromoacetate in NMP. It was incubated in the dark for about 30 minutes, and then 10 μl of 12.3 μl mercaptoglycerol was added. Following an overnight reaction, the solution was desalted with an Amicon Ultra 4 (30 kDa cutoff) to a final volume of about 400 μl.

Next, 50 μl of 1 M sodium acetate at pH 5 was added, followed by 9 μl of 0.5 M sodium periodate. Oxidation was allowed to proceed for 10 minutes in the dark at room temperature. The reaction was then quenched by the addition of 50% glycerol. The low molecular weight components were removed on the same Amicon Ultra 4 device and diafiltered into saline. The final volume was about 200 μl.

The above protocol eliminated one of the desalting steps by adding excess mercaptoglycerol to the solution containing bromoacetylated-DT and bromoacetate.

II. Conjugation

1 ml of amino-oxy Pn14 (−9 mg/ml) was added to the oxidized DT and 100 μl of 1 M sodium acetate at pH 5 added. The reaction was allowed to proceed overnight at room temperature in the dark, and then fractionated on an S400HR column (1×60 cm, equilibrated with saline).

The high molecular weight fraction was pooled. Protein was estimated using 1 mg/ml=1 OD and the Pn14 concentration determined using the resorcinol assay. The fraction was found to contain about 1.3 mg DT/mg Pn14.

### Example 20

### Preparation of a gp350(ox)-AO-S-Pn14 Conjugate

gp350 is a glycoprotein from Epstein Barr virus that binds to human complement receptor. It was produced recombinantly in yeast cells by Dr. Goutam Sen (Uniformed Services University of the Health Sciences, Bethsda, Md.) and purified by hydrophobic interaction chromatography.

The pH of 0.5 ml of gp350 at 8 mg/ml in PBS was reduced by the addition of 50 μl M sodium acetate, pH 4.7, and 11 μl of 0.5 M sodium periodate (in water) was added. After an 8 minute incubation in the dark, on ice, the reaction was quenched by the addition of 100 μl 50% glycerol. Excess reagent was removed by diafiltration using an Amicon Ultra 4 (30 kDa cutoff) device. A total of four, 4 ml exchanges with PBS were used. The final volume was about 300 μl. To this solution, 100 μl of 1 M NaAc, pH 5 was added, followed by 400 μl of AO-S-Pn14 (9.1 mg/ml).

After an overnight reaction at 4° C. in the dark, the reaction was quenched by the addition of 100 μl of 0.25 M amino-oxy acetate, pH 5. The resulting conjugate was fractionated by gel filtration on a 1×60 cm S400HR column, equilibrated with saline. The void volume fractions were pooled. The Pn14 concentration was determined by the resorcinol/sulfuric acid method and the protein from the absorbance at 280 nm, using an extinction coefficient of 1 mg/ml=1 absorbance unit. The conjugate contained 0.9 mg gp350/mg Pn14.

Control gp350 was oxidized and prepared as above but amino-oxy acetate was added instead of amino-oxy Pn14.

In competition assays with fluorescently labeled gp350, both the control and the conjugated gp350 were capable of binding to the complement receptor of human B cells. (Performed by Goutam Sen USUHS).

Mice were immunized on with the gp350-Pn14 conjugate on days 0 and 10, and bled on days 10 and 23

| Day | Anti-Pn14 IgG Titer |
|---|---|
| 10 | 630 |
| 23 | 10643 |

The increase in anti-Pn14 IgG on boosting is an indication that the protein and polysaccharide are covalently linked and acting as a T cell dependent antigen. As a T cell independent antigen, Pn14 alone does not show an increase in titer.

### Example 21

### Preparation of a [DeAcLTA(ox)-AO-SH]-GMBS-BSA Conjugate

LTA was deacylated by incubation for 1 hour in pH 10 sodium bicarbonate at approximately 75° C. Sample is then dialyzed against water. This is deacylated LTA (DeAcLTA).

The sample was then oxidized in 10 mM sodium periodate at pH 5 overnight in the dark at room temperature, dialyzed against water again, and lyophilized. The sample was taken up in a small volume of water, incubated overnight with reduced amino-oxy cysteamine and lyophilized. The sample was taken up in about 1 ml of water and fractionated on an S200HR column, equilibrated with 10 mM sodium acetate, 150 mM NaCl, and 5 mM EDTA, pH 5. The low molecular weight fraction containing both Pi and thiol was pooled and lyophilized and taken up in about 0.75 ml water. This fraction was found to contain about 1 mM thiol and 350 micromolar phosphate. This material is thiol-labeled DeAcLTA.

BSA was labeled with a 50 fold molar excess of GMBS (Prochem) at pH 7.2 and desalted in sodium acetate buffer and concentrated using an Amicon Ultra 4 (30 kDa cutoff) device to a final concentration of about 55 mg/ml.

60 μl of the BSA-GMBS was added to the thiol labeled DeAcLTA. As the reaction proceeded, the concentration of thiols decreased at least 10 fold, as determined by the DTNB assay.

Conjugation was monitored by SEC HPLC on a Superose 6 column, (equilibrated with PBS, 1 ml min, OD 280). The lower trace indicated the chromatogram for the GMBS labeled BSA alone and the upper trace indicated the conjugate, which elutes earlier, indicating an increased molecular weight. By SDS PAGE, the MW of the conjugate had clearly increased in a manner consistent with the SEC chromatogram. No monomeric BSA is evident in the conjugate.

A western blot was performed to demonstrate the presence of LTA on the high molecular weight protein. It is seen that the conjugate was reactive for LTA. Neither BSA nor LTA alone or the combination indicate any high molecular weight LTA.

To further demonstrate the covalent linkage between LTA and BSA, a double ELISA was performed. The ELISA plate was coated with anti-BSA followed by conjugate or LTA, BSA or the combination. Anti-LTA is then applied and the amount bound determined. Thus, only material containing both BSA and LTA would be detected. Only the conjugate was positive in this assay.

Thus by reaction monitoring, molecular weight, western blotting and double ELISA all indicated the formation of a covalent link between the protein and LTA.

### Example 22

### Preparation of a LTA-TT Conjugate

Reagents were obtained from Aldrich. Aminooxyacetylcystamine was prepared by Dr. David Schwartz of Solulink Inc. (San Diego, Calif.). TCEP was purchased from Pierce.

S. Aureus serotype 5 lab strain (MSSA) was grown by Kemp Biotech (Frederick, Md.) in a 100 liter fermenter. Cells were centrifuged, resuspended and centrifuged into aliquots approximating 10 liters of cells. The cell paste was stored at −70° C. An aliquot was thawed and resuspended in 0.1 M sodium citrate, pH 4.7 and disrupted with a Bead Beater (Biospec Products) using 0.1 m zirconium beads. The device was ice cooled and run 1 min on and 1 min off for 4 cycles. The liquid was removed and the beads washed with citrate buffer. Alternatively, cells were treated with 1 mg/ml lysostaphin at pH 5 overnight at 4° C. and then disrupted using a Microfluidizer Model 110Y (Microfluidizer, Newton, Mass.) with 3 passes at 23,000 psi.

I. LTA extraction

LTA was extracted and purified from cell pellets using either the phenol extraction method of Fischer et al., Improved preparation of lipoteichoic acids. Eur J Biochem, 1983. 133(3): p. 523-30, with minor modifications or using the butanol method of Morath et al., Structure-function relationship of cytokine induction by lipoteichoic acid from Staphylococcus aureus. J Exp Med, 2001. 193(3): p. 393-7.

In brief, the disrupted cell suspension was vigorously mixed for 30 min with an equal volume of n-butanol. The solution was then centrifuged for 20 min at 13,000×g. The upper phase (butanol) was removed and the lower, aqueous phase was lyophilized. Initially the butanol phase was re-extracted and the new aqueous phase tested for LTA by ELISA, however an insignificant amount of LTA was recovered. Pellets were resuspended in 25 ml of citrate buffer and frozen. Pellets from several extraction runs were combined, and the disruption/extraction process repeated.

The solubilized extract was filtered using a Whatman 0.45 μm syringe filter (#6894-2504) and loaded onto a 2.6×20 cm Octyl Sepharose column (Pharmacia), equilibrated with 0.1M ammonium acetate in 15% n-propanol, pH 4.7, at a flow rate of 0.5 ml/min. The column was then washed with 0.1 M sodium acetate in 15% n-propanol until the absorbance at 280 nm returned to baseline. The column was then eluted with 25 mM sodium acetate in 40% n-propanol at 4 ml/min and fractions of 8 ml collected. The phosphate containing fracions were pooled and loaded onto a 5 ml Sepharose Q FF column, equilibrated with the same buffer. When the absorbance returned to baseline, the column was eluted with buffer+0.5 M KCl. Phospate containing tubes were pooled, partially lyophilized to reduce the volume and dialyzed against water to remove salts and lyophilized again.

II. Conjugation of LTA to Tetanus Toxoid

LTA was deacylated by incubating 1 ml (10 mg/ml) in 0.1 M sodium carbonate+0.1 M hydroxylamine for 2 hr at 75° C., followed by dialysis against water using a 3.5 kDa cutoff membrane (Pierce). The solution was then lyophilized and taken up in 0.5 ml water. The deacylated LTA was oxidized by the addition of 100 μl 1 M sodium acetate, pH 5 and 125 μl 0.5 M sodium metaperiodate. After 2 hrs the reaction was quenched by the addition of 100 μl 50% glycerol and dialyzed overnight in the dark against water. This material tested positive for aldehydes in the purpald assay (Lee, C. H. and C. E. Frasch, Quantification of bacterial polysaccharides by the purpald assay: measurement of periodate-generated formaldehyde from glycol in the repeating unit Anal Biochem, 2001. 296(1): p. 73-82).

(Amino-oxyacetate)cysteamine was prepared by solubilizing 11 mg Bis(amino-oxyacetate)cystamine in an aqueous solution of 25% NMP. TCEP (17 mg) in 1 M sodium carbonate was added and the solution incubated for 10 min and then passed over a Dowex lx-8 column (1×3 cm), equilibrated with 10 mM Bistris, pH 6. The DTNB positive fractions in the flow through were pooled. The pooled (amino-oxyacetate)cysteamine was assayed for thiols and determined to contain 5.2 μmole SH. The reagent was combined with the oxidized deacylated LTA and incubated overnight in the dark at 4° C. and then dialyzed against 10 mM sodium acetate, 0.15 M sodium chloride, 5 mM EDTA, pH 5 to remove excess reagent. The final solution was 2 ml at 2.5 mM thiol and 25 mM phosphate.

Tetanus toxoid (TT) (obtained from GlaxoSmithKline, Rixensart, Belgium) was labeled with maleimide by adding a 50-fold molar excess of GMBS (0.1 M stock in NMP) to a 14.6 mg/ml solution of TT buffered in 15 M HEPES, 5 mM EDTA, pH 7.3. After a 1 hr reaction, the solution was desalted by diafilitration into 2 M NaCl using an Amicon Ultra 4 (30 kDa cutoff) device, concentrating to a final volume of 0.4 ml. The retained material was DTNB positive.

The thiolated, deacylated LTA was combined with the maleimide-TT under a stream of nitrogen and the pH adjusted to 6.5 by the addition of 0.75 M HEPES, pH 7.3. After sealing and incubating overnight at 4° C., the solution was assayed and determined to still be 2 mM thiol. An additional 7 mg of TT was labeled with maleimide as above, diafiltered and concentrated to 0.5 ml and added to the reaction mixture. Over 30 min the thiol content steadily decreased and at that time the reaction was quenched by the addition of 100 μl of 0.5 M iodoacetamide+100 μl of 1 M sodium carbonate.

The conjugate was purified using size exclusion chromatography on a Superose 6 (Prep grade) column (1×30 cm), equilibrated with saline.

III. Immunization of Mice

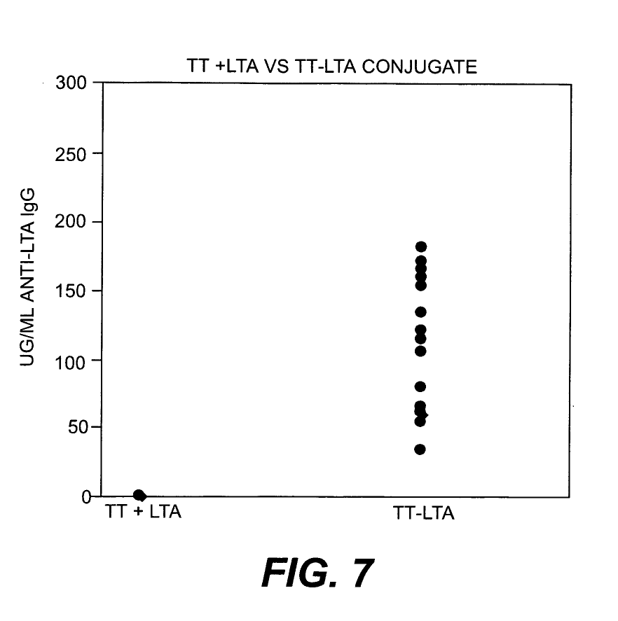

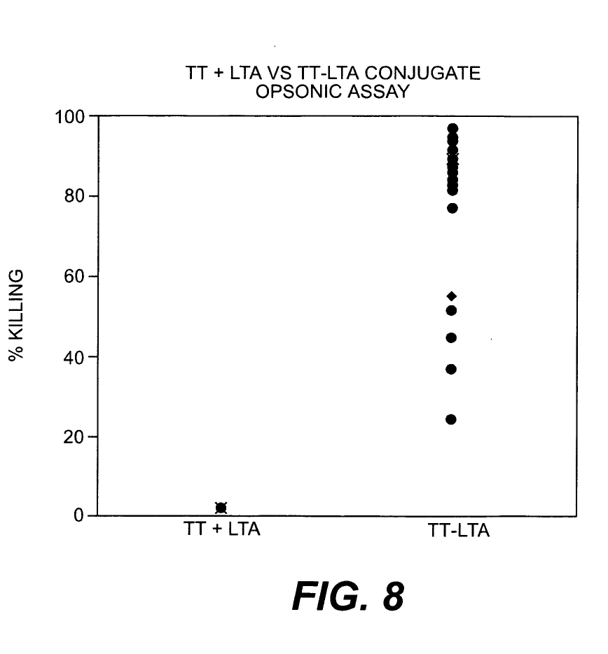

Groups of 20 Balb/c mice were immunized on days 0, 14 and 28 with 5 μg of LTA as described above, either mixed with TT or conjugated to TT and with Ribi MPL adjuvant and bled 14 days later. Individual sera were assayed for anti-IgG by ELISA. The results are provided in FIG. 7. M110 (a mouse monoclonal antibody that binds to LTA) was used as a standard. Anti-LTA levels were assayed in the sera. Results are shown in FIG. 8. The conjugate induced high levels, and the mixture induced only very low levels of antibody. To evaluate the biological activity of the anti-sera, an opsonophagocytic was performed. Sera were diluted at 1:25.

IV. Phosphate Analysis

Phosphate was determined as described by Chen, P. S., T. Y. Toribara, and H. Warner, Microdetermination of Phosphorous. Anal Biochem, (1956) 28: p. 1756-1758, with some modifications. In brief, a 100 μl sample+30 μl magnesium nitrate solution in a 13×100 mm borosilicate tube were vortexed, dried in a heating block and flamed with a propane torch until a brown gas was emitted. 300 μl of 0.5 M HCl was added, the tubes capped with glass marbles and heated in a boiling water bath for 15 min. The tubes were allowed to cool and 700 μl of an ascorbic acid/ammonium molybdate mix added, incubated for 20 min at 45° C. and samples read at 820 nm. The mix was prepared by combining 6 parts of a solution of ammonium molybdate (0.42 g+2.86 ml sulfuric acid made up to 100 ml with water) and 1 part of 10% ascorbic acid in water. Phosphate standard was obtained from Sigma.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. Any of the foregoing process are suitable in accordance with the present invention. The above serve only as illustrative examples and are nonlimiting.

All publications and patents cited in this disclosure are incorporated by reference in their entirety. To the extent the material incorporated by reference contradicts or is inconsistent with this specification, the specification will supercede any such material. The citation of any references herein is not an admission that such references are prior art to the present invention.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

1. A process for preparing a conjugate vaccine, comprising:
(a) reacting a first moiety containing at least one carbonyl group with at least one amino-oxy reagent to form at least one pendent functional group on the first moiety, wherein the first moiety is chosen from polysaccharides, oligosaccharides, carbohydrates, and carbohydrate-containing molecules;
(b) reacting the first moiety containing at least one pendent functional group with a second moiety to form a composition comprising a conjugate, wherein the second moiety is chosen from proteins, peptides, and haptens; and
(c) combining the conjugate with a pharmaceutically acceptable delivery vehicle to form a conjugate vaccine.

2. The process according to claim 1, wherein the at least one carbonyl group is chosen from aldehyde and ketone groups.

3. The process according to claim 1, wherein the amino-oxy reagent is chosen from homofunctional and heterofunctional reagents.

4. The process according to claim 1, wherein the first moiety containing at least one pendent functional group is directly reacted with the second moiety to form a conjugate.

5. The process according to claim 1, wherein the first moiety containing at least one pendent functional group is indirectly reacted with the second moiety to form a conjugate.

6. The process according to claim 1, wherein the second moiety is functionalized prior to its reaction with the first moiety.

7. The process according to claim 1, wherein the protein is a haptenated protein.

8. The process according to claim 1, wherein the carbohydrate-containing molecules are chosen from lipopolysaccharides, lipooligopolysaccharides, lipotechoic acid, and deacylated lipotechoic acid.

9. A process for preparing a conjugate vaccine, comprising:
(a) reacting a first moiety containing at least one pendent amino-oxy group with a second moiety to form a composition comprising a conjugate,
(b) wherein the first moiety is chosen from polysaccharides, oligosaccharides, carbohydrates, and carbohydrate-containing molecules, and the second moiety is chosen from proteins, peptides, and haptens; and
(c) combining the conjugate with a pharmaceutically acceptable delivery vehicle to form a conjugate vaccine.

10. The process according to claim 9, wherein the first moiety containing at least one pendent amino-oxy group is directly reacted with the second moiety to form a conjugate.

11. The process according to claim 9, wherein the first moiety containing at least one pendent amino-oxy group is indirectly reacted with the second moiety to form a conjugate.

12. The process according to claim 9, wherein the second moiety is functionalized prior to its reaction with the first moiety.

13. The process according to claim 9, wherein the protein is a haptenated protein.

14. The process according to claim 9, wherein the carbohydrate-containing molecules are chosen from lipopolysaccharides, lipooligopolysaccharides, lipotechoic acid, and deacylated lipotechoic acid.

15. A process for preparing a conjugate vaccine, comprising:
(a) reacting a first moiety chosen from polysaccharides, oligosaccharides, carbohydrates, and carbohydrate-containing molecules, with
(b) a second moiety reacted with at least one amino-oxy reagent, wherein the second moiety is chosen from proteins, peptides, and haptens, to form a composition comprising a conjugate; and
(c) combining the conjugate with a pharmaceutically acceptable delivery vehicle to form a conjugate vaccine.

16. The process according to claim 15, wherein the amino-oxy reagent is chosen from homofunctional and heterofunctional reagents.

17. The process according to claim 15, wherein the first moiety is directly reacted with the second moiety to form a conjugate.

18. The process according to claim 15, wherein the first moiety is indirectly reacted with the second moiety to form a conjugate.

19. The process according to claim 15, wherein the first moiety is functionalized prior to its reaction with the first moiety.

20. The process according to claim 15, wherein the protein is a haptenated protein.

21. The process according to claim 15, wherein the carbohydrate-containing molecules are chosen from lipopolysaccharides, lipooligopolysaccharides, lipotechoic acid, and deacylated lipotechoic acid.

22. A process for preparing a conjugate vaccine, comprising:
(a) reacting a first moiety with a second moiety containing at least one pendent amino-oxy group to form a composition comprising a conjugate,
wherein the first moiety is chosen from polysaccharides, oligosaccharides, carbohydrates, and carbohydrate-containing molecules, and the second moiety is chosen from proteins, peptides, and haptens; and
(b) combining the conjugate with a pharmaceutically acceptable delivery vehicle to form a conjugate vaccine.

23. The process according to claim 22, wherein the first moiety is directly reacted with the second moiety to form a conjugate.

24. The process according to claim 22, wherein the first moiety is indirectly reacted with the second moiety to form a conjugate.

25. The process according to claim 22, wherein the first moiety is functionalized prior to its reaction with the second moiety.

26. The process according to claim 22, wherein the protein is a haptenated protein.

27. The process according to claim 22, wherein the carbohydrate-containing molecules are chosen from lipopolysaccharides, lipooligopolysaccharides, lipotechoic acid, and deacylated lipotechoic acid.

28. The process according to claim 22, wherein the functionalized second moiety contains at least one pendent amino-oxy group prior to reaction with the first moiety.

29. A process for preparing a conjugate vaccine, comprising:
(a) providing a first moiety chosen from polysaccharides, oligosaccharides, carbohydrates, and carbohydrate-containing molecules;
(b) providing a second moiety chosen from N-terminal 1,2-aminoalcohols containing at least one aldehyde group;
(c) reacting said second moiety with at least one amino-oxy reagent;
(d) reacting said first moiety with the functionalized second moiety to form a composition comprising a conjugate; and
(e) combining the conjugate with a pharmaceutically acceptable delivery vehicle to form a conjugate vaccine.

30. The process according to claim 29, wherein the at least one aldehyde group is created on the second moiety by selective oxidation.

31. The process according to claim 29, wherein the amino-oxy reagent is chosen from homofunctional and heterofunctional reagents.

32. The process according to claim 29, wherein the first moiety is directly reacted with the second moiety to form a conjugate.

33. The process according to claim 29, wherein the first moiety is indirectly reacted with the second moiety to form a conjugate.

34. The process according to claim 29, wherein the first moiety is functionalized prior to its reaction with the second moiety.

35. The process according to claim 29, wherein the carbohydrate-containing molecules are chosen from lipopolysaccharides, lipooligopolysaccharides, lipotechoic acid, and deacylated lipotechoic acid.

36. A process for preparing a conjugate vaccine, comprising:
(a) reacting a first moiety containing at least one pendent amino-oxy group, wherein the first moiety is chosen from polysaccharides, oligosaccharides, carbohydrates, and carbohydrate-containing molecules;
(b) reacting the first moiety with a second moiety to form a composition comprising a conjugate, wherein the second moiety is chosen from glycoproteins containing at least one carbonyl group; and
(c) combining the conjugate with a pharmaceutically acceptable delivery vehicle to form a composition comprising an conjugate vaccine.

37. The process according to claim 36, wherein the at least one carbonyl group is chosen from aldehyde and ketone groups.

38. The process according to claim 36, wherein the first moiety containing at least one pendent amino-oxy group is directly reacted with the second moiety to form a conjugate.

39. The process according to claim 36, wherein the first moiety containing at least one pendent amino-oxy group is indirectly reacted with the second moiety to form a conjugate.

40. The process according to claim 36, wherein the second moiety is functionalized prior to its reaction with the first moiety.

41. The process according to claim 36, wherein the glycoprotein is a haptenated glycoprotein.

42. The process according to claim 36, wherein the carbohydrate-containing molecules are chosen from lipopolysaccharides, lipooligopolysaccharides, lipotechoic acid, and deacylated lipotechoic acid.

43. A process for preparing a conjugate vaccine, comprising:
(a) reacting a first moiety chosen from polysaccharides, oligosaccharides, carbohydrates, and carbohydrate-containing molecules with an amino-oxy reagent, wherein the reaction occurs at a reducing end of the first moiety, to produce a functionalized first moiety;
(b) reacting the functionalized first moiety with a second moiety chosen from proteins, peptides, and haptens to form a composition comprising a conjugate, and
(c) combining the conjugate with a pharmaceutically acceptable delivery vehicle to form a conjugate vaccine.

44. The process according to claim 43, wherein the amino-oxy reagent is chosen from homofunctional and heterofunctional reagents.

45. The process according to claim 43, wherein the first moiety is directly reacted with the second moiety to form a conjugate.

46. The process according to claim 43, wherein the first moiety is indirectly reacted with the second moiety to form a conjugate.

47. The process according to claim 43, wherein the second moiety is functionalized prior to its reaction with the first moiety.

48. The process according to claim 43, wherein the protein is a haptenated protein.

49. The process according to claim 43, wherein the carbohydrate-containing molecules are chosen from lipopolysaccharides, lipooligopolysaccharides, lipotechoic acid, and deacylated lipotechoic acid.

50. A vaccine comprising the conjugate prepared by the process according to claim 1.

51. The use of a vaccine according to claim 1 for preparing a medicament.

52. A process for inducing an immune response in a patient, comprising administering to the patient a vaccine comprising a conjugate prepared according to claim 1.
